(19) United States
(12) Patent Application Publication
(10) Pub. No.: US 2021/0077293 A1
(43) Pub. Date: Mar. 18, 2021

(54) ORAL APPLIANCE AND VALVE ARRANGEMENT

(71) Applicant: Oventus Medical Limited, Indooroopilly, Queensland (AU)

(72) Inventors: David OGLE, Indooroopilly, Queensland (AU); Christopher Patrick HART, Indooroopilly, Queensland (AU); Neil ANDERSON, Indooroopilly, Queensland (AU); Benjamin LOW, Chapel Hill, Queensland (AU); Jonathan CHOI, Indooroopilly, Queensland (AU)

(21) Appl. No.: 16/980,142

(22) PCT Filed: Mar. 12, 2019
(86) PCT No.: PCT/AU2019/050223

§ 371 Date: Sep. 11, 2020

(30) Foreign Application Priority Data
Mar. 12, 2018 (AU) 2018900813
May 2, 2018 (AU) 2018901486
Sep. 26, 2018 (AU) 2018903622

(51) Int. Cl. A61F 5/56; A61M 16/20; A61M 16/06; A61M 16/04; A61M 16/08

(52) U.S. Cl. CPC A61F 5/566; A61M 16/208; A61M 16/1045; A61M 16/049; A61M 16/0875; A61M 16/0666

(57) ABSTRACT

A breathing assistance apparatus includes an oral appliance and a valve arrangement. The oral appliance has an appliance body shaped to be at least partially positioned within the oral cavity of a user. The valve arrangement includes a valve body and a valve member. The valve body includes a valve airway in fluid communication with a user airway, and the valve member is positioned in the valve airway, movable between first and second positions at least in part as a result of airflow through the valve airway. The valve member at least partially obstructs the valve airway in the second position so that there is a differential resistance during inhalation and exhalation.

### FIELD OF THE INVENTION

The present invention relates to a breathing assistance apparatus including an oral appliance and valve arrangement configured to provide a differential resistance during inhalation and exhalation.

### BACKGROUND OF THE INVENTION

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Poor quality or ineffective breathing is an issue which can affect the performance of people in their day to day activities either while they are awake and/or when they are asleep. While awake this can be less optimal performance in activities such as sport or even while performing everyday tasks. While asleep breathing disorders can lead to snoring and/or sleep apnoea.

Snoring arises due to vibration of soft tissues within the respiratory pathways of an individual, and is typically caused by obstructed air movement during breathing while sleeping. Snoring can arise from a range of different physical causes such as blocked sinuses, and typically occurs when the muscles of the upper throat relax during sleep.

Snoring can also be associated with Obstructive Sleep Apnoea (OSA), which is caused by obstruction of the upper airway and results in repetitive pauses in breathing during normal sleep. Individuals having OSA often suffer from daytime sleepiness and fatigue associated with significant levels of sleep disturbance, whilst a partners' sleep patterns are also often disturbed by associated snoring.

Current therapy for treatment of OSA can include lifestyle changes, the use of mechanical devices, such as oral or nasal devices that augment the airway, surgical procedures to enlarge and stabilise the airway during sleep, and continuous or variable positive airway pressure (PAP) devices.

However, surgical procedures can be severe and are not therefore widely used unless absolutely necessary. Whilst PAP devices have had a positive impact, these can be uncomfortable to wear for prolonged time periods, are expensive, and are often noisy, which can in turn lead to additional sleep disturbance. As a result, surgery and PAP treatments have limited application in treating sleep apnoea, and are not generally considered appropriate treatment for snoring.

It has been shown that approximately 30-50% of continuous variable positive airway (CPAP) device users are non-compliant users within 2 years of starting their treatment. CPAP systems deliver airflow to a mask which the user typically wears over their mouth and nose. CPAP masks suffer from several drawbacks including leakage and discomfort and often users experience a degree of claustrophobia whilst wearing the mask.

Furthermore, as CPAP systems must supply air at sufficient pressure to maintain an airway and act as a pneumatic splint, relatively high pressures are typically required. In addition, high flow rates are required as the mask supplies all of the air for a user during inhalation. In order to achieve such high pressures and flow, relatively large and noisy pumps such as air blowers are conventionally used.

It would therefore be desirable to provide a system in which CPAP pressure and/or airflow rates can be minimised in order to reduce noise, vibration and size of pump in order to improve comfort and portability.

In terms of other mechanical devices, nasal devices have been used that dilate the nasal airway using traction or splinting. However, these have typically not had much success and can be uncomfortable for a user.

US2004/194787 describes an anti-snoring device that includes a flexible hollow tube for insertion into the user's mouth, having proximal and distal ends and an outer perimeter. The tube includes an extra oral segment at its proximal end, an intraoral segment at its distal end and an intermediate segment extending therebetween. The extra oral and intraoral segments each include at least one opening. The extra oral segment is for extending beyond the user's outer lips, the intermediate segment is of a sufficient length for extending along the buccopharyngeal pathway of the user's mouth, and the intraoral segment is of a sufficient length for extending beyond a retromolar space in the user's mouth, into the oropharynx and terminating between the posterior tongue and the soft palate. The anti-snoring device also includes a stop extending from the outer perimeter of the tube on the intraoral segment for securing the intraoral segment within the user's oropharynx. However, whilst this arrangement can assist in providing an additional airway, and hence reduce snoring and apnoea events, it can be uncomfortable to wear and can move within the mouth during use, which can reduce device effectiveness and in turn lead to additional breathing problems.

US2005/150504 describes a device which is removably insertable in the mouth for facilitating breathing while sleeping which provides a clear unobstructed airway by protrusive positioning of the mandible and/or delivery of pressurized air to the back of the mouth. The device has upper and lower tooth-contacting members and an airway defined between them, and is designed specifically for use with CPAP machines. Consequently, this device can only be used in limited circumstances, where CPAP machines are available, and is only used in the treatment of sleep apnoea.

WO2012155214 describes an apparatus for providing breathing assistance, the apparatus including a body including a recess for receiving teeth of a user to thereby position the body within an oral cavity of the user, a first opening extending beyond lips of a user to allow air from outside the oral cavity to be drawn in through the opening, a second opening provided in the oral cavity to allow air to be directed into a posterior region of the oral cavity and a channel connecting the first and second openings, the channel extending through at least part of a buccal sulcus of the user.

WO2017/020079 provides an apparatus for providing breathing assistance, the apparatus including a body for positioning within an oral cavity of a user, the body defining, at least one first opening for allowing airflow between lips of the user, two second openings provided in the oral cavity to allow airflow into and out of a posterior region of the oral cavity, two channels, each channel connecting a respective second opening to the at least one first opening and each channel passing at least one of at least partially along the buccal cavity and at least partially between the teeth to thereby provide an airway for the user, the airway at least partially bypassing the nasal passage to thereby act to replicate a healthy nasal passage and pharyngeal space and a tongue retaining portion including a cavity for receiving a portion of a tongue of the user, in use, wherein the tongue retaining portion is configured to retain the tongue in an extended position to project at least partially between the tooth of the user.

### SUMMARY

In one broad form, an aspect of the present invention seeks to provide a breathing assistance apparatus including: an oral appliance having an appliance body shaped to be at least partially positioned within the oral cavity of a user; and, a valve arrangement including: a valve body including a valve airway in fluid communication with a user airway; and, a valve member positioned in the valve airway, the valve member being movable between first and second positions at least in part as a result of airflow through the valve airway and wherein the valve member at least partially obstructs the valve airway in the second position so that there is a differential resistance during inhalation and exhalation.

In one embodiment the oral appliance includes: an appliance airway to allow airflow into and/or out of an oral cavity of the user; and, a nasal airway to allow airflow into and/or out of a nasal cavity of the user.

In one embodiment the valve arrangement generates a positive airway pressure in a user airway by at least one of: restricting airflow during exhalation via the oral cavity; and, restricting airflow during exhalation via the nasal cavity.

In one embodiment the apparatus includes: an oral valve arrangement to control breathing via the oral cavity; and, a nasal valve arrangement to control breathing via the nasal cavity.

In one embodiment a valve configuration is used to control at least one of: a degree of positive airway pressure in a user airway; a degree of airflow during exhalation; and, the differential resistance during inhalation and exhalation.

In one embodiment the valve configuration includes at least one of: a size or number of valve airway openings; a size or number of valve member openings; valve member properties; and, biasing member properties.

In one embodiment the valve configuration is selected based on breathing characteristics of the user.

In one embodiment the valve member includes one or more valve member openings to allow airflow through the valve member when the valve member is in the second position.

In one embodiment the valve body includes one or more openings to allow airflow through the valve body at least when the valve member is in the second position.

In one embodiment movement of the valve member is controlled based on at least one of; valve member properties including at least one of; a valve member thickness; valve member material properties; a valve member stiffness; and, a valve member surface area and, biasing member properties.

In one embodiment movement between the first and second positions is achieved by at least one of; pivotal movement of the valve member; and, deformation of the valve member.

In one embodiment the valve member is a silicone flap.

In one embodiment the valve member is biased into the first position by airflow through the valve airway.

In one embodiment the valve member is biased into the second position by at least one of: airflow through the valve airway; and, elasticity of the valve member.

In one embodiment the valve member is biased into the first position through elastic deformation of the valve member during inhalation and elastically returns to the second position when the user is not inhaling.

In one embodiment the valve member engages the valve body to retain the valve member in the second position.

In one embodiment the appliance body includes an extra-oral opening that extends between lips of the user and an appliance airway passing through the body to an intra-oral opening provided in the oral cavity to allow airflow into and/or out of a posterior region of the oral cavity.

In one embodiment the valve body is coupled to the extra-oral opening through at least one of: a friction fit; an interference fit; a clip fit and, magnetic engagement.

In one embodiment the valve body includes a ring configured to be positioned external to and in abutment with the extra-oral opening, and wherein the valve airway includes one or more valve airway openings through the ring.

In one embodiment the valve member is coupled to the ring so that at least part of the valve member is positioned between the ring and the extra-oral opening.

In one embodiment the valve body includes one or more guides to position the valve body relative to the extra-oral opening.

In one embodiment valve body includes one or more tabs including lips adapted to engage a groove within the extra-oral opening to thereby couple the valve body to the extra-oral opening.

In one embodiment at least part of the valve body is at least one of: shaped to fit within the extra-oral opening; and, shaped to fit over the extra-oral opening.

In one embodiment the valve body includes a hollow tube having a substantially oval cross section.

In one embodiment the valve body includes a number of valve airway openings and wherein the valve member is adapted to obstruct selected ones of the valve airway openings.

In one embodiment the valve arrangement includes a plurality of valve members.

In one embodiment the valve body includes at least one valve airway opening, and wherein the valve member at least partially obstructs the at least one valve airway opening.

In one embodiment the valve body includes a shoulder extending at least part way round the at least one valve airway opening, and wherein the valve member engages the shoulder in the second position to retain the valve member in the second position.

In one embodiment the valve body includes one or more struts extending across the opening, and wherein the valve member engages the struts in the second position to retain the valve member in the second position.

In one embodiment the valve body is coupled to a nasal pillow defining a nasal airway to allow airflow into and out of the nasal cavity of the user.

In one embodiment the nasal pillow is mounted on a nasal pillow connector body attachable to the valve body.

In one embodiment the nasal pillow connector body is movably mounted to the valve body to allow a relative position of the nasal pillow and valve body to be adjusted.

In one embodiment a nasal valve member is positioned in the nasal airway, the nasal valve member being movable between first and second positions at least in part as a result of airflow through the nasal airway and wherein the nasal valve member at least partially obstructs the nasal valve airway in the second position so that there is a differential resistance during inhalation and exhalation via the nasal cavity.

In one embodiment the nasal airway is at least one of: in fluid communication with the valve airway; and, independent of the valve airway.

In one embodiment a device connector is coupled to the valve body, the device connector defining a device airway that supplies airflow from a positive airway pressure device, and wherein the device airway is in fluid communication with at least one of: the valve airway; and, a nasal airway.

In one embodiment the device connector includes a number of device connector airway openings to control air pressure in the device connector airway.

In one embodiment the appliance body includes a hollow lateral base extending inwardly from a hollow arcuate side wall.

In one embodiment an extra-oral opening is defined by a tubular body protruding forwardly from the arcuate side wall.

In one embodiment the appliance body defines at least two channels, each channel connecting an intra-oral opening to an extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space.

In one embodiment the oral appliance includes at least one bite member coupled to the body, the bite member being positioned at least partially between the user's teeth and the body in use.

In one embodiment the at least one bite member mechanically engages an inner surface of the hollow side wall to thereby couple the at least one bite member to the body.

In one embodiment the valve member is part of a valve that is removably mounted to the valve body to allow valve members to be interchanged.

In one embodiment the valve arrangement includes a first valve to control resistance during inhalation and a second valve mechanism to provide resistance during exhalation.

In one embodiment the valve member is mounted on a valve ring, and wherein the valve ring is biased into engagement with a valve seat within the valve body such that movement of the valve member controls resistance during inhalation and biasing of the valve ring against the valve seat controls resistance during exhalation.

In one embodiment the valve arrangement includes: a base that in use is attached to the valve body; a spring; and, a valve ring, the valve ring supporting a valve member movable so that the valve ring is open during inhalation and closed during exhalation, and wherein the valve ring is biased into engagement with a valve seat so that the valve is open during exhalation and closed during inhalation.

In one embodiment the base is movably mounted to the valve body to allow a spring compression to be adjusted and control resistance during exhalation.

In one embodiment the valve arrangement includes: a first valve arrangement to control resistance to airflow via a user's oral cavity; and, a second valve arrangement to control resistance to airflow via a user's nasal cavity.

In one embodiment the apparatus includes a port in communication with at least one of an appliance airway and a valve airway, wherein in use the port is used to deliver at least one of gases and medication to a subject's airway.

In one embodiment the port is coupled to a delivery tube extending along an oral appliance airway.

In one embodiment the apparatus includes a second port in communication with an intra-oral opening, wherein in use the port is used to sample exhaled air.

In one embodiment the apparatus includes first and second ports in communication with respective appliance intra-oral openings.

In one embodiment the appliance body includes a tubular body configured to extend between lips of the user, and wherein the tubular body includes: a first extra-oral opening in fluid communication with an appliance airway passing through the body to an intra-oral opening provided in the oral cavity to allow airflow into and/or out of a posterior region of the oral cavity; and, a second extra-oral opening extending into the oral cavity of the user to provide access to the oral cavity.

In one embodiment the second extra-oral opening is configured to receive a surgical instrument.

In one broad form, an aspect of the present invention seeks to provide a valve arrangement for use with an oral appliance having an appliance body shaped to be at least partially positioned within the oral cavity of a user, the valve arrangement including: a valve body including a valve airway in fluid communication with a user airway; and, a valve member positioned in the valve airway, the valve member being movable between first and second positions at least in part as a result of airflow through the valve airway and wherein the valve member at least partially obstructs the valve airway in the second position so that there is a differential resistance during inhalation and exhalation.

In one broad form, an aspect of the present invention seeks to provide a breathing assistance apparatus including: an oral appliance having an appliance body shaped to be at least partially positioned within the oral cavity of a user, the appliance body including an extra-oral opening that extends between lips of the user and an appliance airway passing through the body to an intra-oral opening provided in the oral cavity to allow airflow into and/or out of a posterior region of the oral cavity; and, a valve arrangement including: a valve body including a valve airway, the valve body being coupled to the extra-oral opening so that the valve airway is in fluid communication with the appliance airway; and, a valve member positioned in the valve airway, the valve member being movable between first and second positions at least in part as a result of airflow through the valve airway and wherein the valve member at least partially obstructs the valve airway in the second position so that there is a differential resistance during inhalation and exhalation.

In one broad form, an aspect of the present invention seeks to provide a valve arrangement for use with an oral appliance having an appliance body shaped to be at least partially positioned within the oral cavity of a user, the appliance body including an extra-oral opening that extends between lips of the user and an appliance airway passing through the body to an intra-oral opening provided in the oral cavity to allow airflow into and/or out of a posterior region of the oral cavity, the valve arrangement including: a valve body including a valve airway, the valve body being coupled to the extra-oral opening so that the valve airway is in fluid communication with the appliance airway; and, a valve member positioned in the valve airway, the valve member being movable between first and second positions at least in part as a result of airflow through the valve airway and wherein the valve member at least partially obstructs the valve airway in the second position so that there is a differential resistance during inhalation and exhalation.

In one broad form, an aspect of the present invention seeks to provide a method of configuring a breathing assistance apparatus for a user, the breathing assistance apparatus including: an oral appliance having an appliance body shaped to be at least partially positioned within the oral cavity of a user; and, a valve arrangement including: a valve body including a valve airway in fluid communication with a user airway; and, a valve member positioned in the valve airway, the valve member being movable between first and second positions at least in part as a result of airflow through the valve airway and wherein the valve member at least partially obstructs the valve airway in the second position so that there is a differential resistance during inhalation and exhalation, the method including determining breathing characteristics of a user; and, configuring the breathing assistance apparatus at least partially in accordance with the breathing characteristics of the user.

In one embodiment the method includes configuring the breathing assistance apparatus by configuring the valve arrangement.

In one embodiment the method includes configuring the valve arrangement to generate a positive airway pressure in a user airway by at least one of restricting airflow during exhalation via the oral cavity; and, restricting airflow during exhalation via the nasal cavity.

In one embodiment the method includes configuring the breathing assistance apparatus to generate a positive airway pressure in a user airway by connecting a device airway to a device connector coupled to the valve body, the device connector defining a device airway that supplies airflow from a positive airway pressure device, and wherein the device airway is in fluid communication with at least one of the valve airway; and, a nasal airway.

In one embodiment the method includes progressively introducing positive airway pressure in a user airway by: restricting airflow during exhalation via the oral cavity; restricting airflow during exhalation via the nasal cavity; restricting airflow during exhalation via the oral and nasal cavities; supplying positive airway pressure from a positive airway pressure device; and, supplying positive airway pressure from a positive airway pressure device and restricting airflow during exhalation via the oral cavity.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which:—

FIG. 1A is a schematic front topside perspective view of an example of an oral appliance:

FIG. 1B is a schematic rear topside perspective view of the oral appliance of FIG. 1A;

FIG. 1C is a schematic front view of the oral appliance of FIG. 1A;

FIG. 1D is a schematic plan view of the oral appliance of FIG. 1A;

FIG. 1E is a schematic cut-away view of the oral appliance along the line A-A′ of FIG. 1D:

FIG. 2A is schematic rear topside perspective view of an example of a valve arrangement;

FIG. 2B is a schematic front topside perspective view of the valve arrangement of FIG. 2A;

FIG. 2C is a schematic front topside perspective view of a breathing assistance apparatus including the oral appliance of FIG. 1A and valve arrangement of FIG. 2A;

FIG. 2D is a schematic front view of the breathing assistance apparatus of FIG. 2B;

FIG. 2E is a schematic side cutaway view of the breathing assistance apparatus of FIG. 2C;

FIG. 2F is a schematic plan cutaway view of the breathing assistance apparatus of FIG. 2C;

FIG. 3A is a schematic rear topside perspective view of a further example of a valve arrangement;

FIG. 3B is a schematic front perspective view of the valve arrangement of FIG. 3A;

FIG. 3C is a schematic front view of the valve arrangement of FIG. 3A;

FIG. 3D is a schematic side view of the valve arrangement of FIG. 3A;

FIG. 3E is a schematic side cutaway view of a breathing assistance apparatus including the oral appliance of FIG. 1A and valve arrangement of FIG. 3A;

FIG. 4A is a schematic rear topside perspective view of a further example of a valve arrangement;

FIG. 4B is a schematic front topside perspective view of the valve arrangement of FIG. 4A;

FIG. 4C is a schematic side cutaway view of the valve arrangement of FIG. 4A;

FIG. 4D is a schematic side cutaway view of a breathing assistance apparatus including the oral appliance of FIG. 1A and valve arrangement of FIG. 4A;

FIG. 5A is a schematic front underside perspective view of a further example of a valve arrangement;

FIG. 5B is a schematic front topside perspective view of a breathing assistance apparatus including the oral appliance of FIG. 1A and valve arrangement of FIG. 5A;

FIG. 5C is a schematic perspective side cutaway view of the breathing assistance apparatus of FIG. 5B;

FIG. 5D is a schematic plan cutaway view of the breathing assistance apparatus of FIG. 5A;

FIG. 6A is a schematic front underside perspective view of a further example of a valve arrangement;

FIG. 6B is a schematic rear topside perspective view of the valve arrangement of FIG. 6A;

FIG. 6C is a schematic side cutaway view of the valve arrangement of FIG. 6A;

FIG. 6D is a schematic side cutaway view of a breathing assistance apparatus including the oral appliance of FIG. 1A and valve arrangement of FIG. 6A;

FIG. 7A is a schematic front topside perspective view of a further example of an oral appliance;

FIG. 7B is a schematic rear topside perspective view of the oral appliance of FIG. 7A;

FIG. 8A is a schematic front topside perspective view of a further example of a breathing assistance apparatus including a valve arrangement;

FIG. 8B is a schematic front topside cutaway view of the breathing assistance apparatus of FIG. 8A;

FIG. 8C is a schematic underside view of the breathing assistance apparatus of FIG. 8A;

FIG. 8D is a schematic side cutaway view of the breathing assistance apparatus of FIG. 8A;

FIG. 9A is a schematic front topside perspective view of a further example of a breathing assistance apparatus including a valve arrangement;

FIG. 9B is a schematic front topside cutaway view of the breathing assistance apparatus of FIG. 9A;

FIG. 10A is a schematic front topside perspective view of a further example of a breathing assistance apparatus;

FIG. 10B is a schematic side cutaway view of the breathing assistance apparatus of FIG. 10A;

FIG. 10C is a schematic underside view of the breathing assistance apparatus of FIG. 10A;

FIG. 11A is a schematic front topside perspective view of a further example of a breathing assistance apparatus:

FIG. 11B is a schematic side cutaway view of the breathing assistance apparatus of FIG. 11A:

FIG. 11C is a schematic underside view of the breathing assistance apparatus of FIG. 11A;

FIG. 12A is a schematic front topside perspective view of an example of a valve arrangement:

FIG. 12B is a schematic side cutaway view of the valve arrangement of FIG. 12A;

FIG. 13A is a schematic front topside perspective view of a further example of a breathing assistance apparatus;

FIG. 13B is a schematic side cutaway view of the breathing assistance apparatus of FIG. 13A;

FIG. 14A is a schematic front topside perspective view of a further example of a valve arrangement for a breathing assistance apparatus:

FIG. 14B is a schematic perspective view of a nasal pillow of FIG. 14A;

FIG. 15A is a schematic front topside perspective view of a further example of a breathing assistance apparatus;

FIG. 15B is a schematic perspective view of a nasal pillow of FIG. 15A;

FIG. 16A is a schematic front topside perspective view of a further example of a valve arrangement for a breathing assistance apparatus:

FIG. 16B is a schematic perspective cutaway view of the valve arrangement of FIG. 16A:

FIG. 16C is a schematic perspective cutaway view of the valve arrangement of FIG. 16A;

FIG. 17A is a schematic front underside perspective view of a further example of a valve arrangement for a breathing assistance apparatus;

FIG. 17B is a schematic perspective cutaway view of the valve arrangement of FIG. 17A;

FIG. 18A is a schematic front underside perspective view of a further example of a valve arrangement for a breathing assistance apparatus:

FIG. 18B is a schematic perspective cutaway view of the valve arrangement of FIG. 18A:

FIG. 18C is a schematic front top view of a valve adjuster:

FIG. 18D is a schematic front underside perspective view of the valve arrangement of FIG. 18A and the valve adjuster of FIG. 18C;

FIG. 19A is a schematic front topside perspective view of a further example of a valve arrangement for a breathing assistance apparatus:

FIG. 19B is a schematic perspective cutaway view of the valve arrangement of FIG. 19A:

FIG. 20A is a schematic front topside perspective view of an example of a connector arrangement;

FIG. 20B is a schematic rear underside view of the connector arrangement of FIG. 20A;

FIG. 20C is a schematic front topside perspective view of an example of an oral appliance incorporating the connector arrangement of FIG. 20A:

FIG. 20D is a schematic rear topside perspective view of the oral appliance of FIG. 20C;

FIG. 20E is a schematic plan cutaway view of the oral appliance of FIG. 20C:

FIG. 21A is a schematic front underside perspective view of a further example of a connector arrangement:

FIG. 21B is a schematic rear underside view of the connector arrangement of FIG. 21A;

FIG. 22A is a schematic front topside perspective view of a further example of a connector arrangement:

FIG. 22B is a schematic rear underside view of the connector arrangement of FIG. 22A;

FIG. 22C is a schematic transverse cutaway front topside perspective view of the connector arrangement of FIG. 22A;

FIG. 22D is a schematic sagittal cutaway rear underside perspective view of the connector arrangement of FIG. 22A;

FIG. 22E is a schematic front topside perspective view of the connector arrangement of FIG. 22A attached to a front port;

FIG. 23A is a schematic front topside perspective view of an example of an oral appliance;

FIG. 23B is a schematic rear topside perspective view of the oral appliance of FIG. 23A;

FIG. 23C is a schematic front topside perspective view of the oral appliance of FIG. 22A attached to the connector arrangement of FIG. 22E;

FIG. 23D is a schematic rear topside perspective view of the oral appliance of FIG. 22C;

FIG. 23E is a schematic sagittal cutaway front topside perspective view of the oral appliance of FIG. 22C;

FIG. 24A is a schematic front topside perspective view of an example of an oral appliance;

FIG. 24B is a schematic rear topside perspective view of the oral appliance of FIG. 23A;

FIG. 25A is a schematic front topside perspective view of the oral appliance of FIG. 24A attached to an alternative example of a connector;

FIG. 25B is a schematic sagittal cutaway front topside perspective view of the oral appliance of FIG. 25A:

FIG. 26A is a schematic front topside perspective view of an inline valve arrangement and connector;

FIG. 26B is a schematic sagittal cutaway front topside perspective view of the inline valve of FIG. 26A;

FIG. 27A is a schematic front topside perspective view of an example of an oral appliance;

FIG. 27B is a schematic rear topside perspective view of the oral appliance of FIG. 27A;

FIG. 27C is a schematic front view of the oral appliance of FIG. 27A;

FIG. 27D is a schematic rear view of the oral appliance of FIG. 27A;

FIG. 27E is a schematic cut-away view of the oral appliance along the line B-B′ of FIG. 27C;

FIG. 27F is a schematic cut-away view of the oral appliance along the line C-C′ of FIG. 27C;

FIG. 28A is a schematic front topside perspective view of an example of an oral appliance;

FIG. 28B is a schematic rear topside perspective view of the oral appliance of FIG. 28A;

FIG. 28C is a schematic transverse cutaway rear topside perspective view of the oral appliance of FIG. 28A:

FIG. 28D is a schematic transverse cutaway rear topside perspective view of a modified version of the oral appliance of FIG. 28A;

FIG. 29A is a schematic front perspective view of a further example of a valve arrangement for a breathing assistance apparatus;

FIG. 29B is a schematic front cutaway perspective view of the valve arrangement of FIG. 29A;

FIG. 30A is a front view of an example of a nasal pillow connector for use in a valve arrangement in accordance with the present disclosure;

FIG. 30B is a schematic cutaway view of the nasal pillow connector of FIG. 31A;

FIG. 30C is a schematic topside view of the nasal pillow connector of FIG. 31A;

FIG. 31A is a schematic front perspective view of a further example of a valve arrangement for a breathing assistance apparatus:

FIG. 31B is a schematic front cutaway perspective view of the valve arrangement of FIG. 31A;

FIG. 32A is a schematic front perspective view of a further example of a valve arrangement for a breathing assistance apparatus;

FIG. 32B is a schematic front cutaway perspective view of the valve arrangement of FIG. 32A:

FIG. 32C is a schematic front view of the valve arrangement of FIG. 32A;

FIG. 32D is a schematic side view of the valve arrangement of FIG. 32A;

FIG. 32E is a schematic cutaway side view of the valve arrangement of FIG. 32A; and,

FIG. 33 is a schematic front perspective view of a further example of a valve arrangement for a breathing assistance apparatus.

### DETAILED DESCRIPTION OF THE EMBODIMENTS

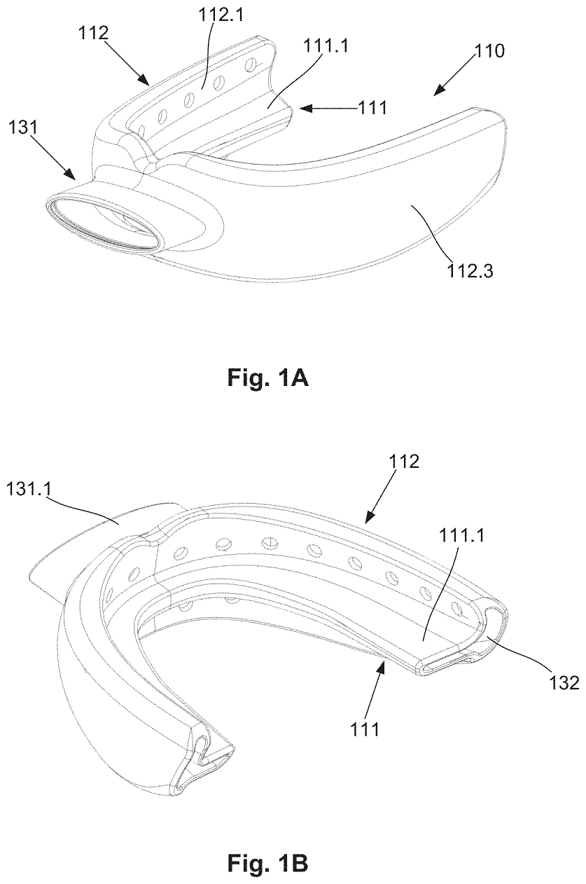

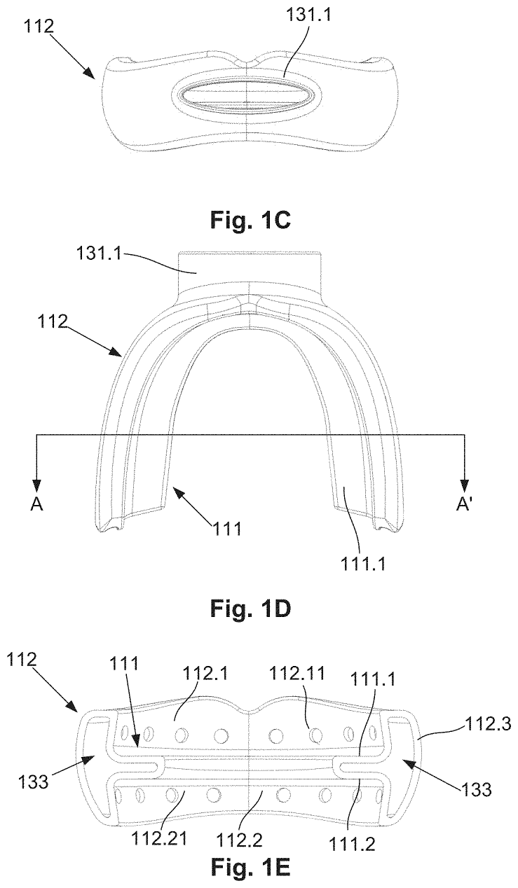

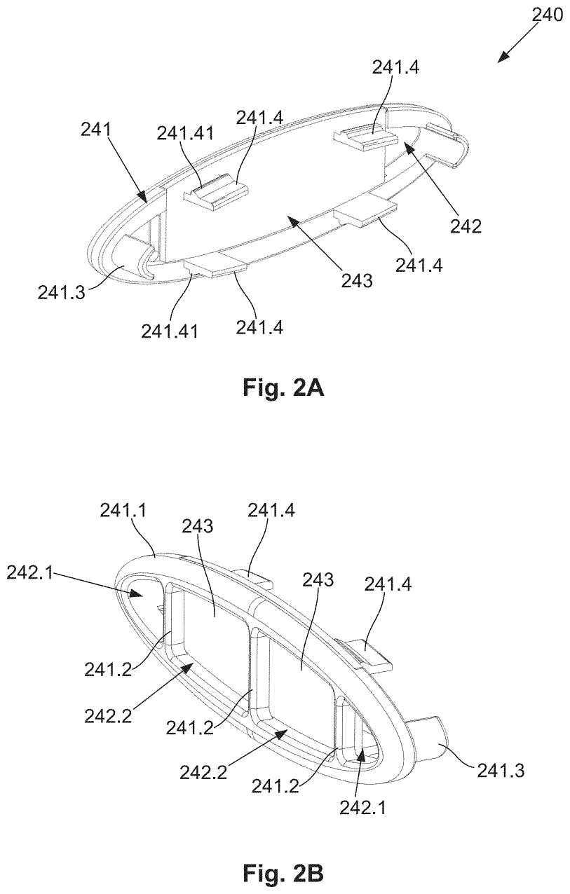

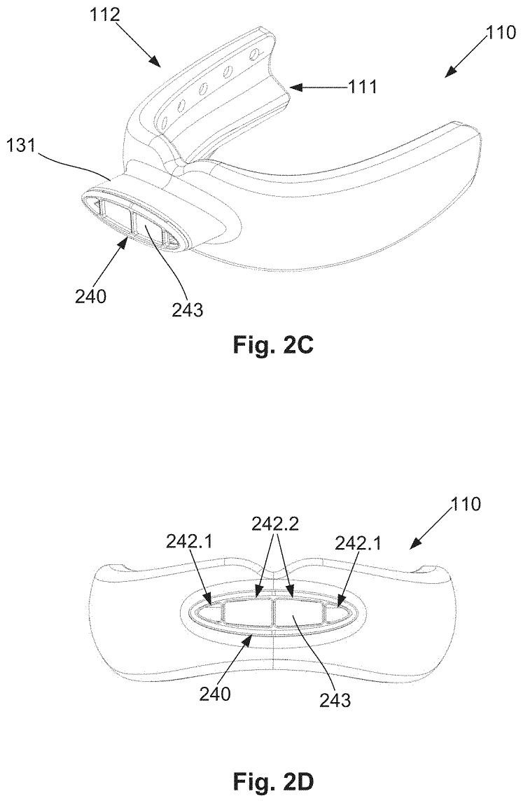

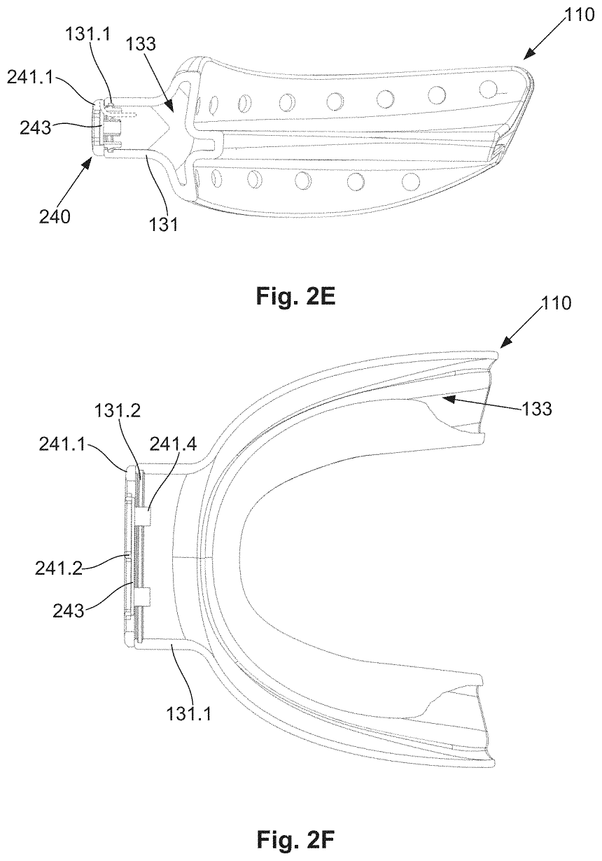

An example of a breathing assistance apparatus will now be described with reference to FIGS. 1A-1E and 2A-2F.

In particular, an oral appliance is shown in FIGS. 1A to 1E. The oral appliance includes an appliance body 110 shaped to be at least partially positioned within the oral cavity of a user. The appliance body includes an extra-oral opening 131 that extends between lips of the user, and an appliance airway 133 passing through the body 110, to one or more intraoral openings 132 provided in the oral cavity, to allow airflow into and out of a posterior region of the oral cavity.

Specifically in this example, the oral appliance includes a hollow lateral base 111, having spaced upper and lower surfaces 111.1, 111.2, extending inwardly from a hollow arcuate side wall 112, having upper and lower inner side walls 112.1, 112.2 spaced from a curved outer side wall 112.3. In this example, the extra-oral opening 131 includes a tubular body 131.1 that protrudes forwardly from the arcuate side wall 112.3, with this being in fluid communication with the appliance airway 133, which extends through the hollow base 111 and side wall 112.

In use, the base 111 is positioned between the user's maxillary and mandibular teeth, with the arcuate side wall 112 positioned between the user's teeth and cheeks, and the tubular body 131.1 extending between the user's lips so that the airway is accommodated at least partially between the teeth and in the buccal cavity between the teeth and cheeks. This allows air to pass through the appliance airway so that it is directed into a posterior region of the mouth through the second openings 132, thereby at least partially bypassing the nasal passage and acting to replicate a healthy nasal passage and pharyngeal space.

Providing airflow directly into a posterior portion of the user's oral cavity has a number of benefits. In particular, this avoids obstructions created by the nasal cavity, soft palate and tongue which can lead to snoring and apnoea events, and helps reduce the drying effects of airflow, which can in turn lead to user discomfort. Furthermore, providing the airway between the teeth and through the buccal cavity allows a significant cross sectional size to be accommodated within the oral cavity without undue discomfort. This makes the apparatus comfortable to wear whilst ensuring an unobstructed airflow thereby preventing snoring and apnoea events. Thus, for example, nasal obstructions can be bypassed by airflow through the apparatus, thereby bypassing the nasal airway or adding to it in the case of a partial obstruction. Furthermore, air flowing below or on both sides of the soft palette helps prevent collapse of the soft palate, which can in turn lead to additional obstruction.

In one example, the body 110 is made of metal and in particular a titanium alloy and/or cobalt chromium alloy. However, it will be appreciated that any suitable material may be used, including high strength polymers, plastics, VeroGlaze (MED620) dental material, or the like. This can be achieved using additive printing, injection moulding or any other suitable technique. For example, the body 110 can be manufactured using laser sintering of a nylon material, or injection moulding of a polymer, such as a thermosetting polymer, a thermoplastic polymer, silicone, an elastomer, polyvinylsiloxane, polyurethane, ethylvinylacetate, polycarbonate, acrylonitrile butadiene styrene, or a combination of these materials.

The body 110 can be coated with a medical grade polymer and in one example, a medical grade elastomer, such as silicone or polyurethane, epoxy or parylene, for improved comfort as well as ensuring biocompatability. In one example, the coating can include an Active Composite Guidance, which is a three-dimensional composite resin with different shapes and sizes and which can be bonded to the body to ensure accurate positioning of the body with respect to the user's teeth. Coatings can be applied to the body using any suitable technique, such as dip coating, vapour coating, or spray coating the body, thereby ensuring all exposed surfaces, including internal surfaces of the channels, are coated. As part of this process, this can include applying primers to the body prior to coating, thereby ensuring the coating adheres to the body. As an alternative, or in addition to coating, at least part of the body can be polished using at least one of mechanical and electrochemical polishing.

In one example, the body is custom made by measuring the oral cavity of the user, for example by taking dental impressions, a series of photos, or scans of the user's teeth and/or oral cavity and then customising the apparatus based on the measured size, as will be described in more detail below. However, more typically a range of standard sized bodies can be produced, with an appropriate body being selected based on a closest fit to the intended user. Custom fitting can then be achieved using inserts positioned between the user's teeth and the body in use. Each insert is typically customised for a user's teeth and is adapted to be removable and/or replaceable.

Inserts can also be made by injection moulding similar materials to the body, could be made by additive manufacturing, such as 3D printing, and/or could be manufactured by having the user bite into a material that can be moulded to the shape of the user's teeth and then solidified. For example, this could include UV curing, using a thermosetting material or the like. In one example, the inserts are formed from boil and bite materials, such as Ethylene-vinyl acetate or the like, although silicone or other materials, such as a thermosetting polymer, a thermoplastic polymer, silicone, an elastomer, polyvinylsiloxane, polyurethane, ethylvinylacetate, polycarbonate, acrylonitrile butadiene styrene, or a combination of these materials, could be used. For example, this could be used to allow a user to mould the inserts at home by biting into a bite into a member made of a suitable material such as silicone.

In one example, the apparatus can be used be with a plurality of different inserts, which can be used for example to provide different levels of fit, comfort, support or the like. The inserts can also be either temporary or semi-permanent, and may be made from different materials depending on their intended use. For example, a temporary insert could be created upon the initial fitting of a breathing assist apparatus, using a silicone which is moulded in-situ, with this being replaced by a subsequent semi-permanent insert, such as a 3D printed acrylic insert, once there has been opportunity for this to be manufactured. This allows an initial fitting to be performed when the apparatus is initially supplied with temporary inserts, with semi-permanent acrylic inserts being subsequently manufactured and provided to the user once ready.

Whilst the inserts could be fitted using any suitable technique, in one example, the inserts can be attached to the first and second bodies using adhesives, mechanical couplings, such as interference fits, or the like. In this regard, in this example, the upper and lower inner side walls 112.1, 112.2 include a number of openings 112.1, 112.21 which are used to mechanically engage the inserts (not shown). In particular, the inserts attached to the upper and lower side walls 112.1, 112.2, positioned in abutment with the upper and lower base surfaces 111.1, 111.2, and optionally bonded using an adhesive.

The use of inserts, as described above, allows a variation in the shape of teeth and jaws that can be accommodated by the body, allowing the majority of individuals to be fitted by selecting one of a number of predefined template bodies, having standard sizes/dimensions.

Additionally, inserts can be manufactured so as to be thermoformable, allowing these to be reshaped slightly by heating to accommodate changes in the user's jaw positioning or shape over time. Even such semi-permanent inserts would typically undergo wear and potential discolouration, and hence can be replaced periodically. Despite this, the first and second bodies can be reused as needed, so inserts can be recreated from the previously scanned moulds. The ability to remove inserts allows these to be replaced and/or cleaned and re-used as required. Similarly, the bodies can also be cleaned and/or sterilised prior to re-use.

In the above example, the body 110 is shown as a single unitary body. However, this is not essential and alternatively the body 110 can be manufactured as separate upper and lower bodies, which engage separately with the upper and lower jaw. This allows different sizes of body to be used, as well as allowing a relative position of the upper and lower bodies to be adjusted and thereby adjust a degree of mandibular advancement.

In this regard, it is known that mandibular advancement can assist in holding open the user's airway, which in turn can reduce snoring. For example, temporomandibular joint disorder (TMD) arises when the upper and lower jaws are misaligned. This may be naturally occurring or can result from injury, or the like. Regardless, such jaw misalignment tends to contribute to airway obstructions by changing the shape of the upper airway, and moving the tongue towards the posterior of the oral cavity, which can in turn exacerbate issues associated with OSA and snoring. Accordingly, by allowing the relative position of the first and second bodies to be adjusted, this allows the jaws of the user to be aligned thereby reducing the effects of TMD, and hence further reducing the likelihood of snoring and OSA.

The oral appliance of FIGS. 1A to 1E can be used in conjunction with a valve arrangement and an example valve arrangement, and its operation, will now be described with reference to FIGS. 2A to 2F.

In this example the valve arrangement 240 includes a valve body 241 including a valve airway 242. The valve body 241 is coupled to the extra-oral opening 131 of the oral appliance body 110, as shown in FIGS. 2C to 2F, so that the valve airway 242 is in fluid communication with the appliance airway 133.

A valve member 243 is positioned in the valve airway 242, and is moveable between first and second positions at least in part as a result of airflow through the valve airway 242. In the second position, shown in FIGS. 2C to 2F, the valve member 243 at least partially obstructs the valve airway 242, whereas the obstruction is removed or at least reduced when the valve member 243 is in a first position (shown in dotted lines in FIG. 2E). As a result, there is a differential resistance during inhalation and exhalation.

Accordingly, the above described arrangement provides a valve arrangement which can be coupled to an oral appliance, such as the oral appliance of FIGS. 1A to 1E, to adapt the oral appliance so that that oral appliance may introduce differential resistance during inhalation and exhalation.

In one example, the valve arrangement is configured so that airway resistance is minimal during inhalation, thereby maximising airflow into the user's lungs, whilst introducing a greater resistance during exhalation, which in turn can help maintain pressure within the user's airway. This can assist in regulating breathing and in particular allow for rapid inhalation, whilst ensuring slower exhalation, thereby maintaining a minimum pressure within the system to prevent collapse of the airway and optimising gas exchange within the lungs, for example to minimise the chances of hyperventilation. The ability to maintain internal airway pressure can assist in maintaining an open airway, which in turn can avoid the need for the application of external PAP.

Accordingly, this allows the system to be used not only for snoring, but also for the treatment of sleep apnoea in all severities of patients who would otherwise require to be treated with positive airway pressure and/or to receive supplemental air and/or oxygen.

It will also be appreciated that other configurations could be provided, such as to increase resistance during inhalation, which can be useful as a means for providing breathing exercises for a user.

In the above example, the valve body is separate to, and designed to couple to the oral appliance, allowing the oral appliance to be used without the valve, or to allow different valve arrangements to be interchanged. However, this is not essential and alternatively, the valve arrangement can be integrally formed with the oral appliance. In this instance, the valve body would be formed by part of the oral appliance body. It will therefore be appreciated that reference to an appliance body and a valve body is not intended to exclude arrangements in which the valve arrangement is integral with the appliance.

A number of further features will now be described.

In the above example, the oral appliance includes an appliance airway to allow airflow into and/or out of an oral cavity of the user. However, this is not essential, and additionally and/or alternatively, the oral appliance could include a nasal airway to allow airflow into and/or out of a nasal cavity of the user, and examples of such arrangements will be described in more detail below. It will therefore be appreciated that the provision of an appliance airway is not essential, and that fluid communication between the valve and user airway could be achieved via other mechanisms, such as via an airway external to and/or separate from the oral appliance. Accordingly, reference to the appliance airway in the above example should not be considered as necessarily restrictive.

As mentioned above, in one example, the valve arrangement generates a positive airway pressure in a user's airway by restricting airflow during exhalation via the oral cavity and/or restricting airflow during exhalation via the nasal cavity. A positive airway pressure in the user's airway can also be achieved by connecting a device airway to a device connector coupled to the valve body. In this case, the device connector provides a device airway that supplies airflow from a positive airway pressure device, with the device airway being in fluid communication with either the valve airway or a nasal airway, allowing positive pressure to be supplied to the user.

Accordingly, it will be appreciated that the apparatus can be configured to create a positive airway pressure, using a variety of techniques, with the technique used typically depending on breathing characteristics of the user, and in particular a severity of breathing difficulties. For example, if the user is not experiencing major breathing difficulties, the oral appliance can be used with the airway only, in order to facilitate airflow during inhalation and exhalation. However, if airway collapse is a risk for the user, the above described mechanisms can be used to maintain progressively higher positive airway pressures, until airway collapse is mitigated and/or prevented.

Thus, typically a potential user would be assessed and then trialled with the oral appliance only. If this did not address issues, mechanisms would be used to maintain progressively higher positive airway pressures until satisfactory results are achieved. This will typically involve initially restricting airflow during exhalation via the oral cavity only. If this fails then airflow during exhalation via the nasal cavity is restricted, with these mechanisms being used in conjunction in the event they are unsuccessful alone. If the issues are still not addressed, positive airway pressure would be supplied from a positive airway pressure device, with this being further performed in conjunction with restricting airflow during exhalation via the oral cavity in the event that supplied positive pressure alone did not address the issue. Thus, different mechanisms can be trialled successively, until a desired positive airway pressure is maintained.

As will become apparent from the following description, each of the mechanisms can be implemented using the oral appliance used in conjunction with a valve arrangement. This can avoid the need for users to require the use of a mask, as required by traditional PAP machines, which is typically uncomfortable, and of limited effectiveness in some cases. Additionally, as masks can leak, the use of mask-based PAP systems can lead to requirements for increased airflow, which in turn requires higher powered pumps, which tend to be noisy, and in turn lead to disturbed sleep. In contrast, delivering positive pressure via the following arrangements allows air to be directed more effectively into the user airway, either via nasal or appliance airways, in turn reducing the airflow requirements needed to achieve desired results, whilst avoiding the discomfort associated with a mask.

The apparatus can include an oral valve arrangement to control breathing via the oral cavity and/or a nasal valve arrangement to control breathing via the nasal cavity. Thus, depending the severity of the user's condition, either or both of oral and nasal valve arrangements can be used.

In one example, the apparatus is further controllable by controlling a valve configuration. In particular, this can be used to control differential resistance during inhalation and exhalation, which in turn influences a degree of positive airway pressure in a user airway and/or a degree of airflow during exhalation. Thus, in addition to controlling the mechanisms used to deliver positive airway pressure, the extent of the delivered pressure can be controlled by adjusting the valve configuration for each mechanism, and this can again be performed in accordance with selected breathing characteristics of the user.

In this regard, the valve configurations can include a size or number of valve airway openings, a size or number of valve member openings, valve member properties or biasing member properties.

For example, the valve member can include one or more valve member openings to allow airflow through the valve member when the valve member is in the second (closed) position, allowing exhalation to occur while the valve member is closed. In this example, the size and number of openings will control the level of resistance during exhalation, in turn controlling the resulting positive airway pressure in the user airway. Similarly, the valve body can include one or more openings to allow airflow through the valve body at least when the valve member is in the second (closed) position, with the number and size of the openings again controlling the positive airway pressure. The valve member can be configured to only partially close openings in the airway, in which case the extent to which the openings remain open will influence the positive airway pressure.

Finally, movement of the valve member can be controlled to adjust the relative ease and speed with which the valve opens or closes, which in turn can help control the build-up of the positive airway pressure, and in particular the rate at which this commences and ceases. Factors influencing the valve opening or closing can include any one or more of valve member properties, such as a valve member thickness, valve member material properties, a valve member stiffness, a valve member resilience or elasticity, a valve member surface area, or the like. Additionally and/or alternatively, this could depend on biasing member properties, for example if a biasing member is used to urge the valve into the second position.

A number of further more specific features will now be described.

Movement of the valve member 243 between the first and second positions can be achieved in a variety of manners depending upon the preferred implementation. In the example of FIGS. 2A to 2F, the valve member 243 is at least partially flexible, allowing movement of the valve member 243 to be achieved through deformation of the valve member. In one preferred example the valve member 243 is a silicone flap which can be deformed at least in part as a result of airflow through the airway 242. However, this is not essential, and additionally and/or alternatively movement could be achieved through pivotal or hinged movement of the valve member, for example if the valve member 243 is a rigid body.

The valve member 243 is typically biased into the first position by airflow through the valve airway 242 and then biased into the second position by airflow in an opposing direction through the valve airway 242 and/or through elasticity of the valve member 243. Tus, in the current example, the valve member 243 is biased into the first position through elastic deformation of the valve member 243 during inhalation, with the valve member returning to the second position elastically when the user is not inhaling.

Furthermore, the valve member 243 typically engages the valve body 241 to retain the valve member in the second position, thereby preventing further movement of the valve member during exhalation, for example to prevent the valve blowing outwardly.

The valve body 241 could be coupled to the extra-oral opening in a variety of manners. Typically this is achieved in a reversible manner, to allow the valve body 241 to be attached to and then subsequently removed from the extra-oral opening, and can involve the use of a friction fit, an interference fit, a clip fit or through magnetic engagement. Such arrangements allow the valve arrangement to be coupled to or decoupled from the oral appliance, in turn allowing valve arrangements to be interchanged, for example allowing different levels of resistance to be introduced depending on user requirements. In this regard, it will be appreciated that if the valve member 243 is suitably configured, obstruction of the airway could be complete obstruction, forcing the user to breathe out entirely through their nose. More typically however, the valve member 243 is sized to provide a partial obstruction allowing exhalation to occur through the valve arrangement, whilst maintaining a positive airway pressure within the patient's airway to maintain airway inflation.

In the example shown in FIGS. 2A and 2B, the valve body 241 includes a ring 241.1 which is adapted to be provided external to and in abutment with the extra-oral opening, and in particular an end of the tubular body 131.1. The valve airway includes one or more openings 242 through the ring 241.1, with two outer and two inner openings 242.1, 242.2 being shown in this example, delineated by vertical struts 241.2 extending between upper and lower halves of the ring 241.1.

The valve body 241 typically includes guides to position the valve body relative to the extra-oral opening. Specifically, in this example, the guides 241.3 are arc segments that extend rearwardly from the ring 241.1 and act to engage an inner surface of the tubular body 131.1, thereby positioning the valve body 240 relative to the extra-oral opening.

The valve body 241.1 further includes a number of tabs 241.4, which in this example extend rearwardly from the ring 241.1, the tabs 241.4 include lips 241.41 which engage a groove 131.2 within the extra-oral opening to thereby couple the valve body to the extra-oral opening using a clip fit configuration. In this example, the groove extends circumferentially around an inner surface of the tubular body 131.1, although it will be appreciated that alternative configurations could be used, for example having the groove around only part of the tubular body 131.1, or having the groove on an outer surface of the tubular body 131.1.

In the current example, the valve member 243 is in the form of a silicone flap, coupled to the ring 241.1, and in particular to an upper part of the ring 241.1. The coupling can be achieved through adhesive bonding, welding, or the like, or could involve the use of mechanical engagement, such as clipping or attachment using fasteners. This could be performed in a reversible manner, allowing the valve member 243 to be removed and replaced as needed. In any event, in this example part of the valve member 243 is positioned between the ring 241.1 and the tubular body 131.1 to help further secure the valve member 243 in position, whilst allowing the valve member 243 to deform by upward deflection during inhalation. This allows airflow through each of the inner and outer openings 242.1, 242.2 during inhalation. In contrast, during exhalation the valve member 243 returns to the position shown in FIG. 2A, with the valve member 243 abutting against the vertical struts 241.2, thereby obstructing the inner openings 242.2 and restricting airflow to the outer openings 242.1 during exhalation.

In the above described example, the valve member includes a single valve member which is formed as a naturally planar body provided in abutment with the struts 241.2 so that the valve member 243 is urged into engagement with the struts 241.2 during exhalation to retain the valve member in the second position during exhalation. However, it will be appreciated that other configurations could be used.

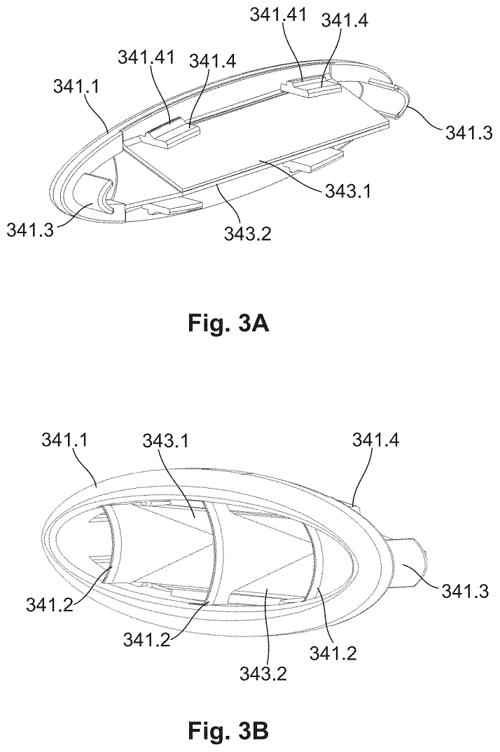

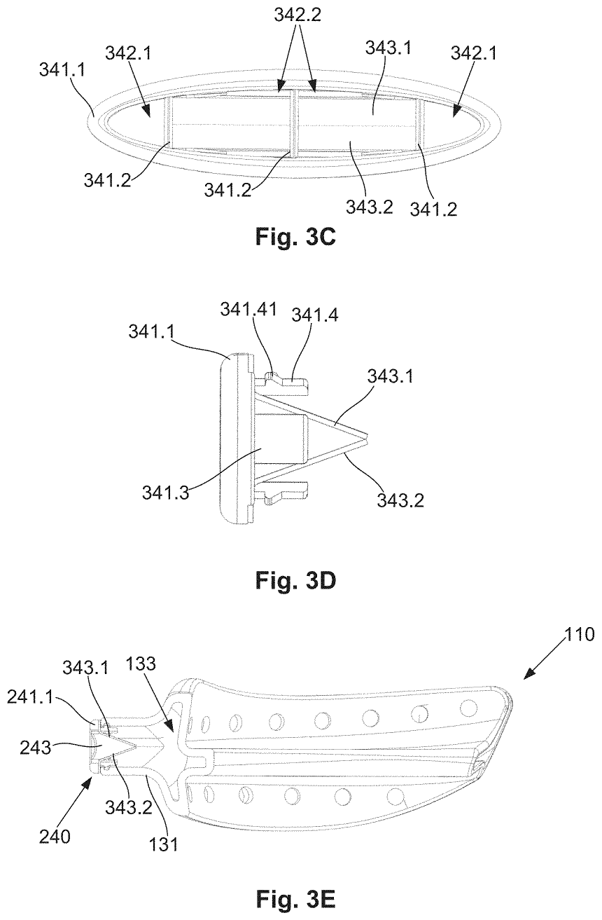

An example of an alternative configuration is shown in FIGS. 3A to 3D. This arrangement is substantially similar to that described above with respect to FIGS. 2A to 2D, with similar reference numerals increased by 100 denoting similar features, which will not therefore be described in further detail.

In contrast to the previous example, this valve arrangement includes two valve members 343.1, 343.2, which are provided in a substantially V-shaped configuration, supported by triangular trailing edges of struts 341.2. The valve members 343.1, 343.2 are positioned in abutment at an apex, so that as the user inhales the valve members 343.1, 343.2 hinge apart to allow airflow therebetween. As inhalation ceases, the valve members 343.1, 343.2 return to the V-shaped configuration blocking inner openings 342.2, so that exhalation is via outer openings 342.1 only.

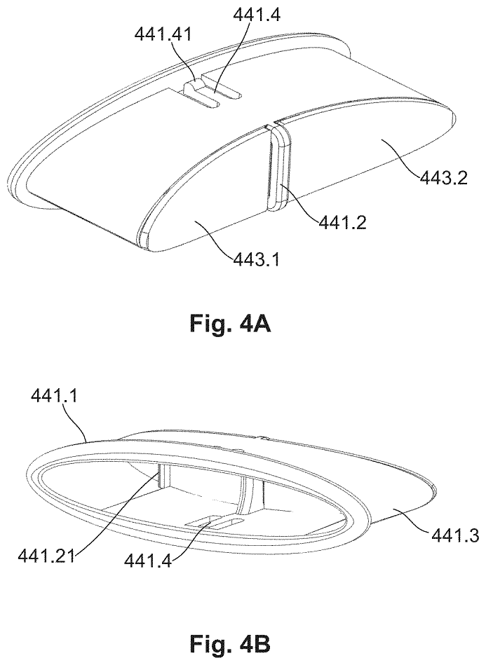

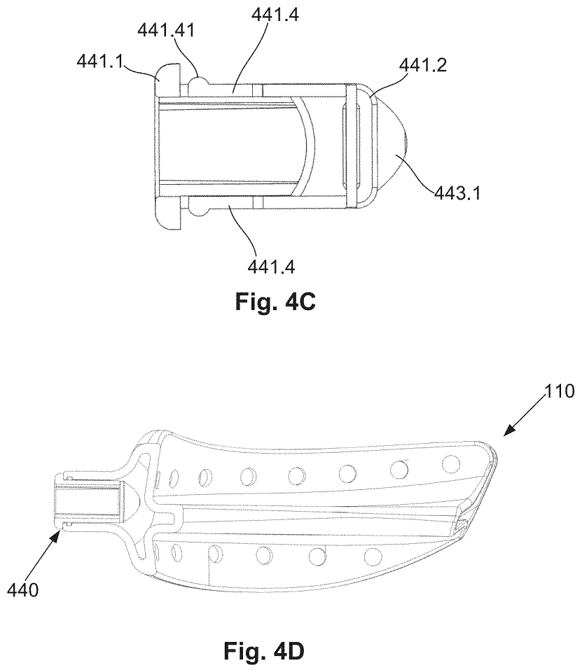

A further example arrangement is shown in FIGS. 4A to 4D.

In this example, the valve body 440 includes a ring 441.1 and a hollow valve tube 441.3 that extends rearwardly from the ring to act as a guide to position the body. In this example, the hollow valve tube 441.3 has a substantially oval cross-section that is shaped to fit within the tubular body 131.1 of the extra-oral opening body, although it will be appreciated that other shapes could be used for different shapes of extra-oral opening. The hollow valve tube 441.3 includes tabs 441.4 having lips 441.41 that again engage the groove 131.2 provided within the tubular body 131.1 to help couple the valve body to the extra-oral opening.

In this example, the valve arrangement includes a valve member having two halves 443.1, 443.2 mounted to a central vertical strut 441.2 extending across a centre of a rear of the hollow tube 441.3, with the valve member halves 443.1, 443.2 in abutment with outer vertical struts 441.21 and a rear surface of the hollow tube 441.3 in a rest (second) position. This arrangement allows the valve member halves 443.1, 443.2 to deform rearwardly at outer edges during inhalation, to allow airflow through the tube, returning to the second position during exhalation. It will be appreciated that in this example, the entire airway is substantially blocked by valve member halves 443.1, 443.2, which collectively have a shape corresponding to the cross-sectional area of the hollow tube 441.3. However, this is not essential and shortened versions of the valve member halves 443.1, 443.2 could be used to allow for partial obstruction during exhalation.

In the above examples, the valve arrangement is mounted at least partially within the tubular body 131.1 of the extra-oral opening. However, this is not essential and in alternative configurations, the valve arrangement can be provided external to the tubular body 131.1.

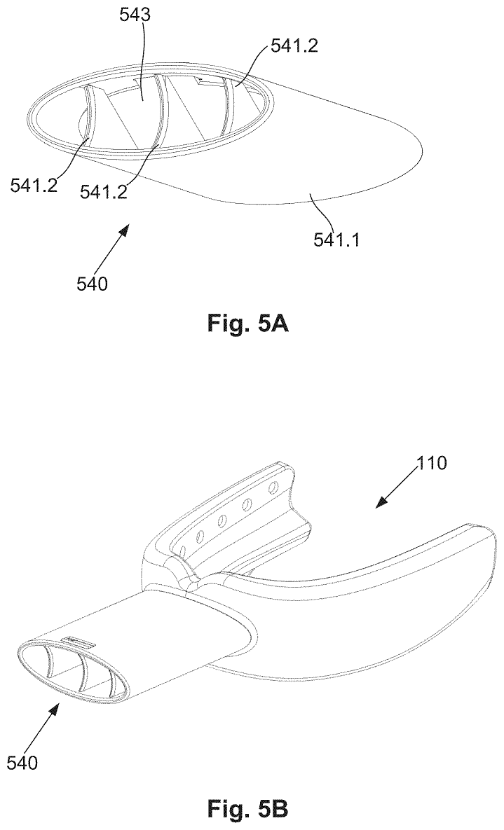

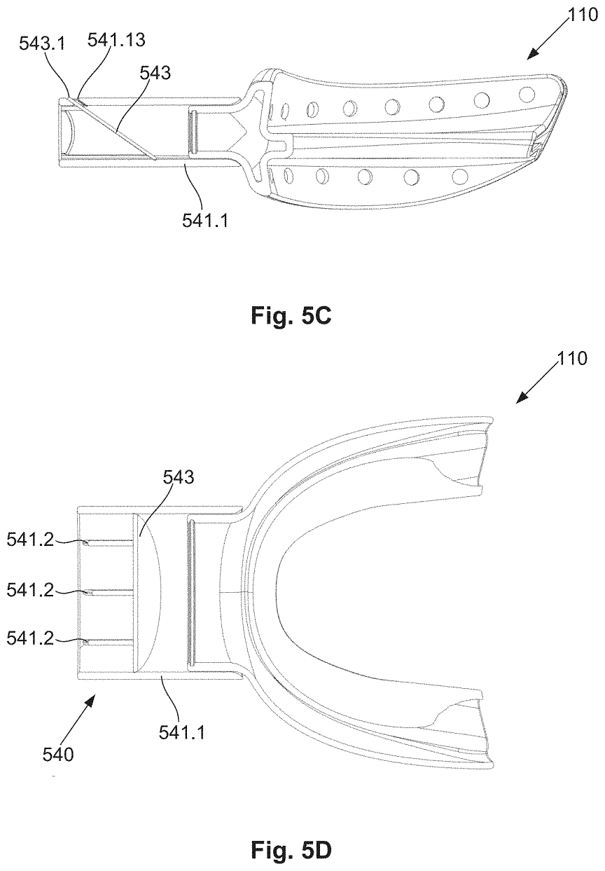

A further example valve arrangement will now be described with reference to FIGS. 5A to 5D.

In this example, the valve arrangement 540 includes a hollow valve tube 541.1 shaped to fit over the tubular body 1131.1 of the extra-oral opening 131. The hollow valve tube 541.1 includes struts 541.2 extending vertically across a mouth of the hollow valve tube 541.1, which, as in previous examples, can be used to define inner and outer openings in the mouth. A valve member 543 includes a tab 543.1 that is mounted in an opening 541.13 in the hollow valve tube 541.1, so that the valve member 543 abuts against a diagonal rearward edge of the struts 541.2.

In use inhalation causes the valve member 543 to bend upwardly, allowing inhalation, whilst during exhalation the flap returns to the position shown in FIG. 5C, thereby obstructing the airway.

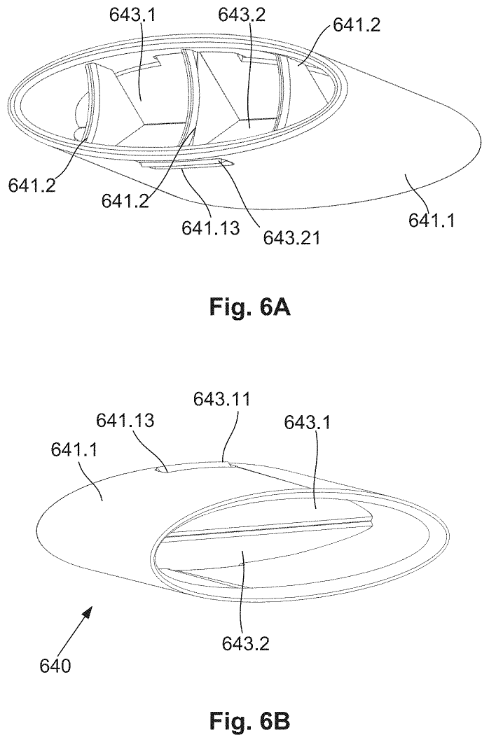

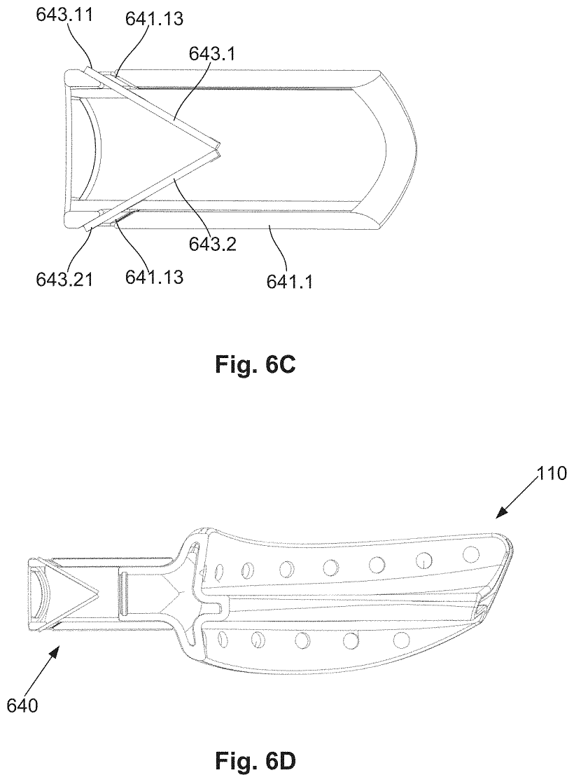

A further variation is shown in FIGS. 6A to 6D. This arrangement is substantially similar to that described above with respect to FIGS. 5A to 5D, with similar reference numerals increased by 100 denoting similar features, which will not therefore be described in further detail.

In contrast to the previous example, this valve arrangement includes two valve members 643.1, 643.2, which are provided in a substantially V-shaped configuration, supported by triangular trailing edges of struts 641.2. The valve members 643.1, 643.2 are positioned in abutment at an apex, so that as the user inhales the valve members 643.1, 643.2 hinge apart to allow airflow therebetween. As inhalation ceases, the valve members 643.1, 643.2 return to the V-shaped configuration blocking inner openings 642.2, so that exhalation is via outer openings 642.1 only.

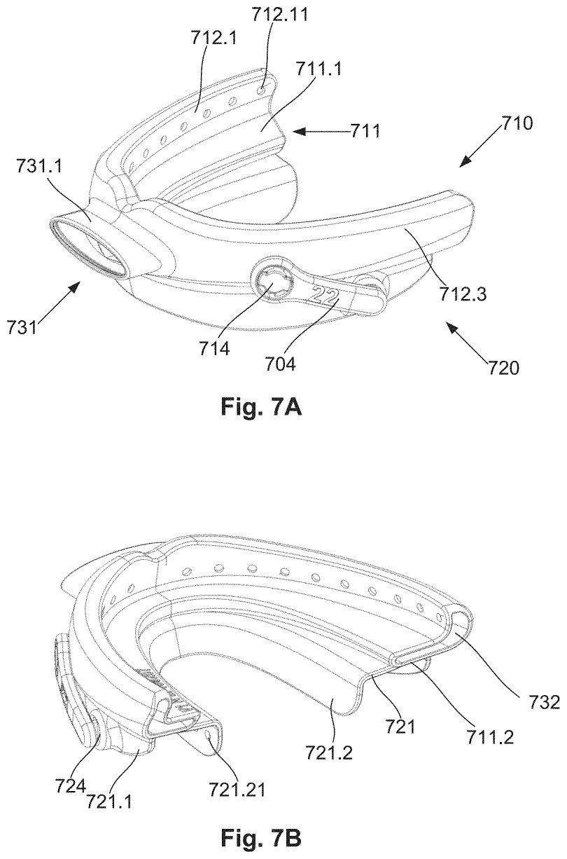

An alternative example of an oral appliance will now be described with reference to FIGS. 7A and 7B.

In this example, the oral appliance includes upper and lower bodies 710, 720. The upper body 710 includes a hollow lateral base 711, having spaced upper and lower surfaces 711.1, 711.2, extending inwardly from a hollow arcuate side wall 712, having an upper inner side wall 712.1 spaced from a curved outer side wall 712.3. The walls define an airway (not shown), terminating in intra-oral openings 732 and connecting to an extra-oral opening 731 formed from a tubular body 731.1 projecting forwardly from the curved outer side wall 712.3. The second body includes a lateral top 721, extending between inner and outer downwardly projecting side walls 722.1, 722.2.

The upper and lower bodies 710, 720 are used with respective bite members, which receive the maxillary and mandibular teeth respectively. The bite members are attached to the bodies 710, 720, typically via mechanical or chemical bonding, for example through the use of plugs that fit within sockets 712.11, 721.21 in the side walls 712.1, 721.2.

In use, the bodies 710, 720 are coupled together so that collectively these act in a manner similar to the body 110 of FIGS. 1A to 1E. However, in this example a relative position of the first and second bodies 710, 720 can be adjusted to control a degree of mandibular advancement. Whilst this can be achieved in any manner, in one example the bodies 710, 720 include respective first and second mountings in the form of lugs 714, 724 that project outwardly from sides of the first and second bodies 710, 720, with these being interconnected via respective arms 704. A relative position of the first and second bodies 710, 720 can then be adjusted based on a length of the arms 704, with the arms 704 being extendible, or interchangeable, allowing different lengths of arms to be provided to achieve a desired degree of mandibular advancement as required.

In the current example, the arms 704 are made of moulded plastic or nylon, with the arms including fittings at each end, which engage with the lugs, typically using an interference fit, such as a locking engagement, or an elastic loop that stretches over the lugs.

A number of further example valve arrangements will now be described. For the purpose of illustration, these are shown with reference to the oral appliance of FIGS. 7A and 7B, but it will be appreciated that the oral appliances of FIGS. 1 and 7 can be used interchangeably and this is not intended to be limiting.

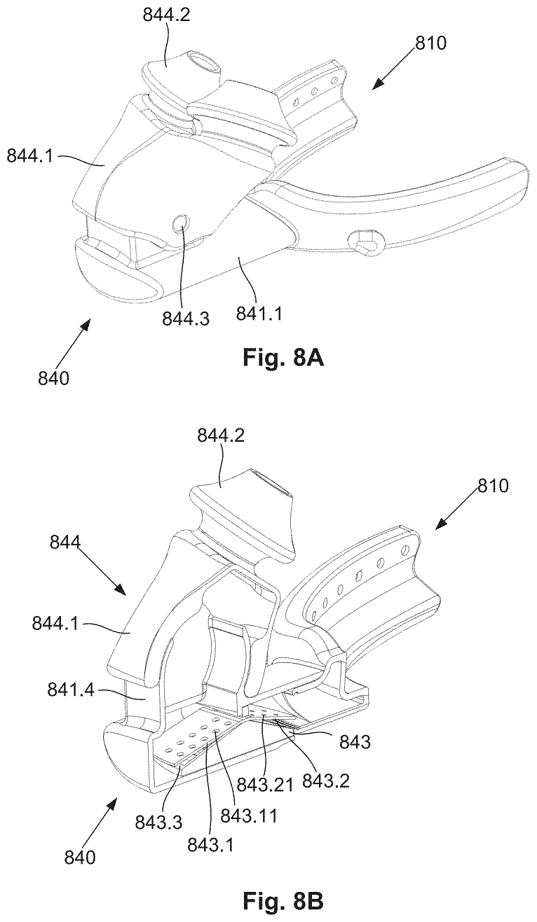

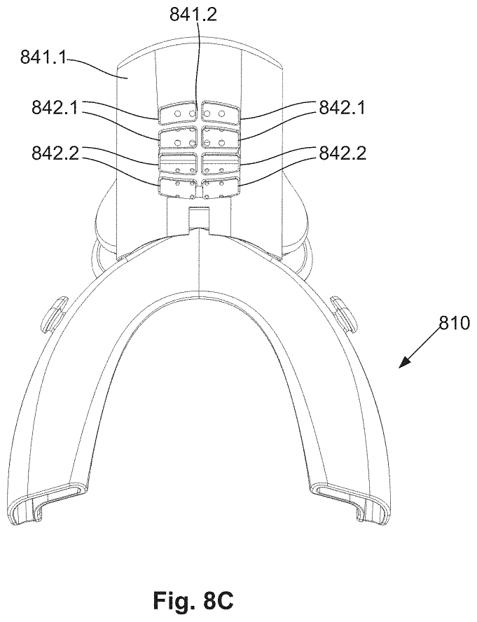

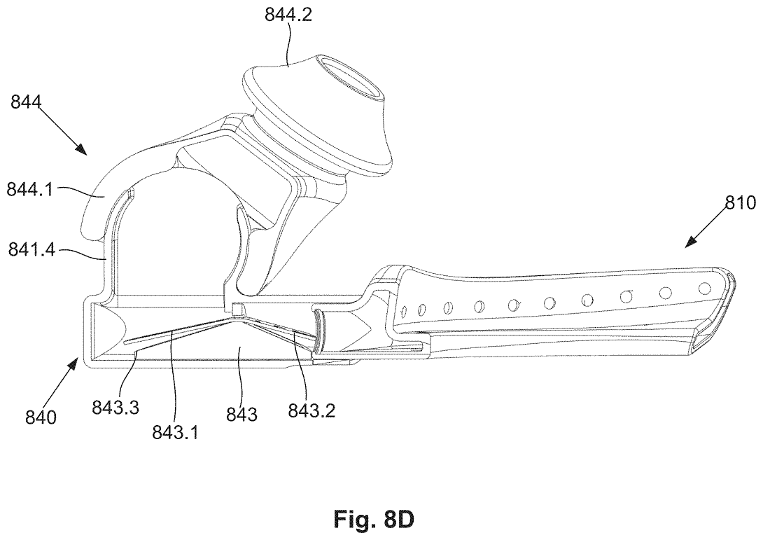

A further example of a valve arrangement will now be described with reference to FIGS. 8A to 8D. The valve arrangement is configured to further provide a nasal airway, via a nasal pillow, although it will be appreciated that certain features are also applicable to the previous examples.

In this example, the valve arrangement includes a hollow tube shaped valve body 841.1 shaped to fit over the tubular body 731.1 of the extra-oral opening 731. The valve body 841.1 is also coupled to a nasal pillow 844.2 defining a nasal airway to allow airflow into and out of the nasal cavity of the user.

In this example, the nasal pillow 844.2 is attached to a nasal pillow connector body 844.1, which is in turn attachable to the valve body 841.1, via a pillow mounting 841.4, which projects upwardly from the valve body 841.1. The pillow mounting 841.4 and nasal pillow connector body 844.1 are shaped to form a cylindrical cup and socket connection, allowing rotation of the nasal pillow connector body 844.1 about a pivotal pin 844.3. This allows an angle of the nasal pillow 844.2 relative to the valve body 841.1 to be adjusted, so that in use the nasal pillow 844.2 is aligned with the user's nose, when the valve body 841.1 is mounted to the oral appliance in use.

At least one valve airway opening 842.1, 842.2 is provided on an underside of the valve body 841.1. In this example, the openings include a nasal opening 842.1, which is in fluid communication with the nasal airway, allowing airflow via the user's nasal cavity, and a valve airway opening 842.2, in communication with the valve airway, allowing airflow via the user's oral cavity. The nasal and valve airways are physically separated, allowing independent airflow through the nasal and valve airways, although it will be appreciated that this is not essential and alternatively, the airways could be in fluid communication to allow airflow therebetween.

A respective valve member 843.1, 843.2 at least partially obstructs nasal openings 842.1 and valve airway openings 842.2 to thereby provide differential airway resistance during inhalation and exhalation as previously described.

In this example, struts 842.2 extending across the openings, to effectively provide multiple nasal openings 842.1 and valve airway openings 842.2. In one example, the valve members 843.1, 843.2 can engage the struts 841.2 in the second position to retain the valve member in the second position during exhalation. Additionally, and/or alternatively, a shoulder 843.3 can be provided extending at least part-way round the at least one valve airway opening, with the valve member engaging the shoulder 843.3 in the second position to retain the valve member in the second position. In this example, the shoulder 843.3 is provided by a frame 843 that supports the valve members 843.1, 843.2, although this is not essential and the shoulder 843.3 could alternatively form part of the valve body 841.1.

In use inhalation causes the valve members 843.1, 843.2 to bend upwardly, allowing unrestricted airflow through the openings 842.1, 842.2, whilst during exhalation the valve members 843.1, 843.2 rest on the shoulder 843.3, thereby obstructing the airways. The degree of obstruction could be controlled by only covering some of the multiple nasal openings 842.1 and/or valve airway openings 842.2. Alternatively, in this example, the valve members 843.1, 843.2 include one or more openings 843.11, 843.21 therein to allow airflow through the valve members 843.1, 843.2 when they are in the second position. Thus, in this configuration, this allows exhalation to occur via the openings 843.11, 843.21, which thereby introduce resistance during exhalation, in turn creating pressure in the oral and nasal cavities, which can help maintain the user's airway open.

It will be appreciated that with this configuration, the size and number of openings 843.11, 843.21 in the valve members 843.1, 843.2 can be used to control pressure during exhalation. Accordingly, by using different numbers and/or sizes of openings 843.11, 843.21, this can be used to relatively adjust the pressure in the nasal and oral cavities during exhalation. Similarly, the size, material, material properties, and/or thickness of each valve member 843.1, 843.2 can be different to adjust the opening force required. For example, a thinner valve member, a valve member with a reduced resilience, stiffness or elasticity, or a valve member with a larger surface area typically requires a lower pressure to open. Accordingly, appropriate configuration of the valve members 843.1, 843.2 and openings 843.11, 843.21, allows the relative degree of inflow and outflow resistance to breathing via the user's nasal and oral airways to be controlled.

In one particular example, the frame 843 on which the valve members 843.1, 843.2 are mounted, is removably mounted within the valve body 841.1, allowing the valve members 843.1, 843.2 to be easily interchanged. This allows a number of different valve members 843.1, 843.2 to be trialled using the same device, allowing an optimum relative degree of inflow and outflow resistance via the nasal and oral cavities to be achieved.

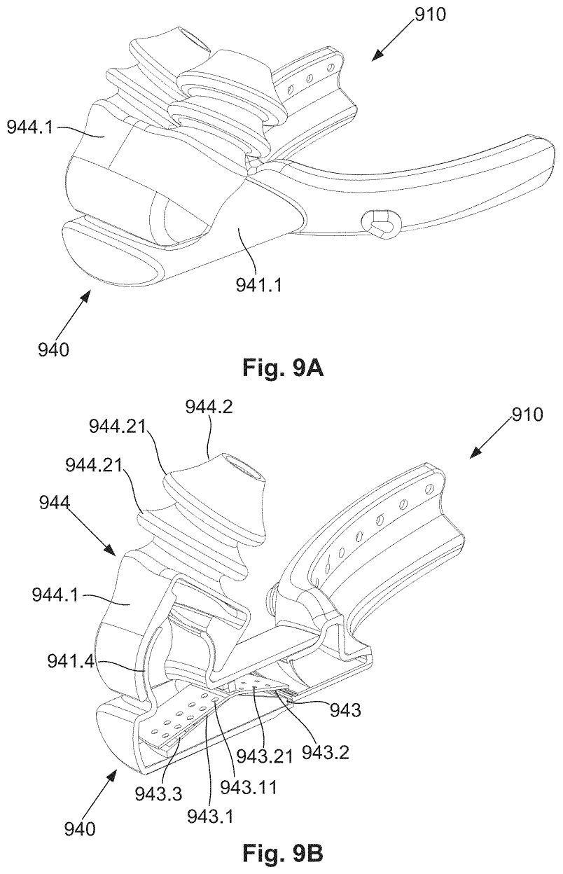

An alternative example will now be described with reference to FIGS. 9A and 9B. For the purpose of illustration similar features to those shown in FIGS. 8A to 8D are used to represent similar features, albeit increased in value by 100, and these will not be described in further detail.

Accordingly, in this example, the valve arrangement includes a hollow tube shaped valve body 941.1 shaped to fit over the tubular body 731.1 of the extra-oral opening 731. The valve body 941.1 also includes a mounting 941.1 coupled to a nasal pillow connector body 944.1 having a nasal pillow 944.2 mounted thereon. In this example, the nasal pillow 944.2 includes a double set of bellows 944.21 to allow for greater compression, in particular allow up to ˜20 mm variation in length, to allow different sizes of nose to be accommodated with greater comfort and fit.

It will be appreciated that this example also includes other similar features, including nasal and valve openings, and valve members 943.1, 943.2, including openings 943.11, 943.21, which are mounted on a frame 943, and which are movable between a first open position and second closed position. Operation is therefore substantially similar to that of the previous example and will not be described in further detail.

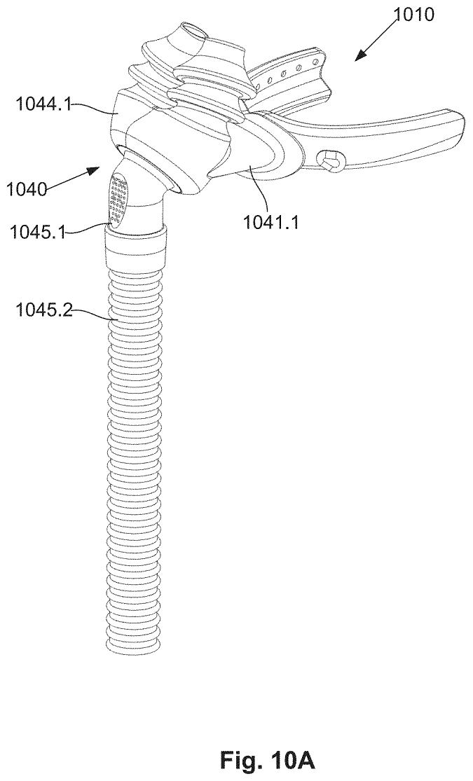

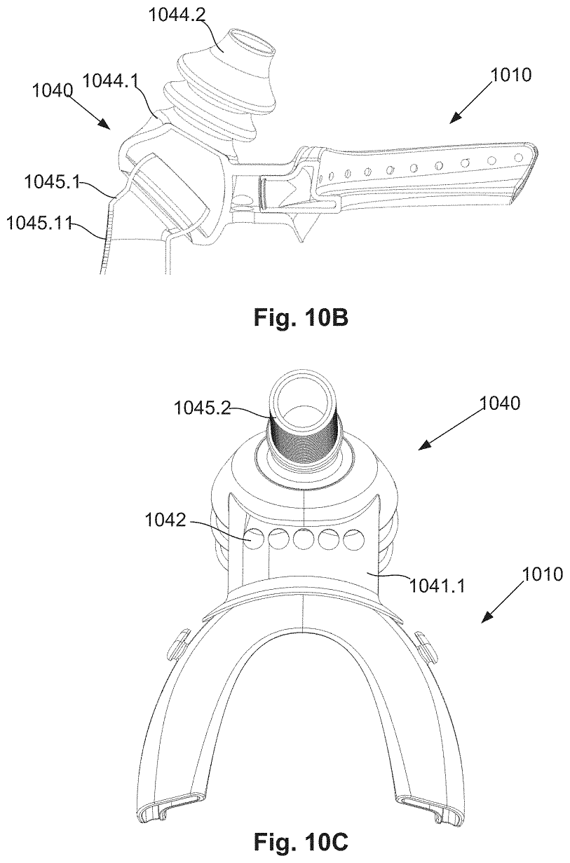

An alternative example will now be described with reference to FIGS. 10A to 10C. For the purpose of illustration similar features to those shown in FIGS. 8A to 8D are used to represent similar features, albeit increased in value by 200, and these will not be described in further detail.

In this example, the valve arrangement includes a hollow tube shaped valve body 1041.1 shaped to fit over the tubular body 731.1 of the extra-oral opening 731. The valve body 1041.1 also includes valve openings 1042 in an underside of the valve body 1041.1. The valve openings 1042 may be used in conjunction with a valve member (not shown) for providing differential resistance during inhalation or exhalation, although this is not essential.

In this example, the valve body 1041.1 includes an integrated nasal pillow connector body 1044.1 having a nasal pillow 1044.2 mounted thereon, with the nasal pillow 1044.2 again including a double set of bellows to allow for greater comfort and fit. The arrangement further includes a device connector 1045.1 coupled to a tube 1045.2 defining a device airway that supplies airflow from a positive airway pressure device. In this example, the device airway is in fluid communication with the nasal airway, allowing positive air pressure to be supplied to the user's nasal cavity. A number of device connector airway openings 1045.11 are provided in the device connector 1045.1 to control air pressure in the device connector airway. Again the openings could be used in conjunction with a valve member to assist in controlling air pressure during inhalation and exhalation.

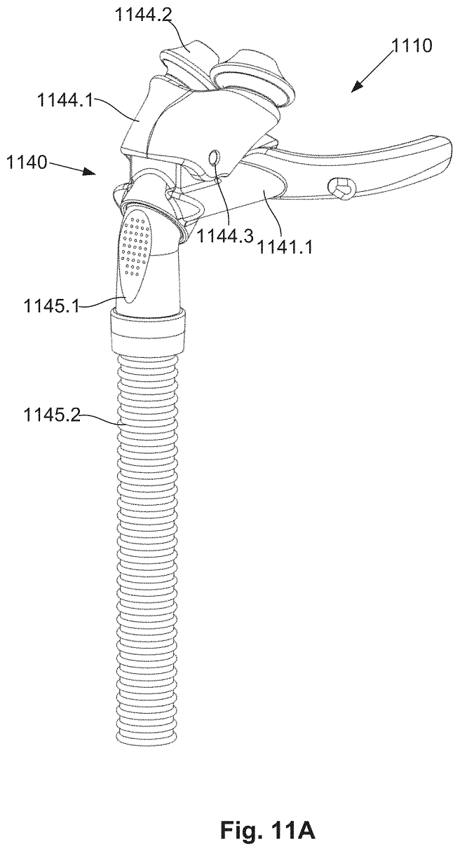

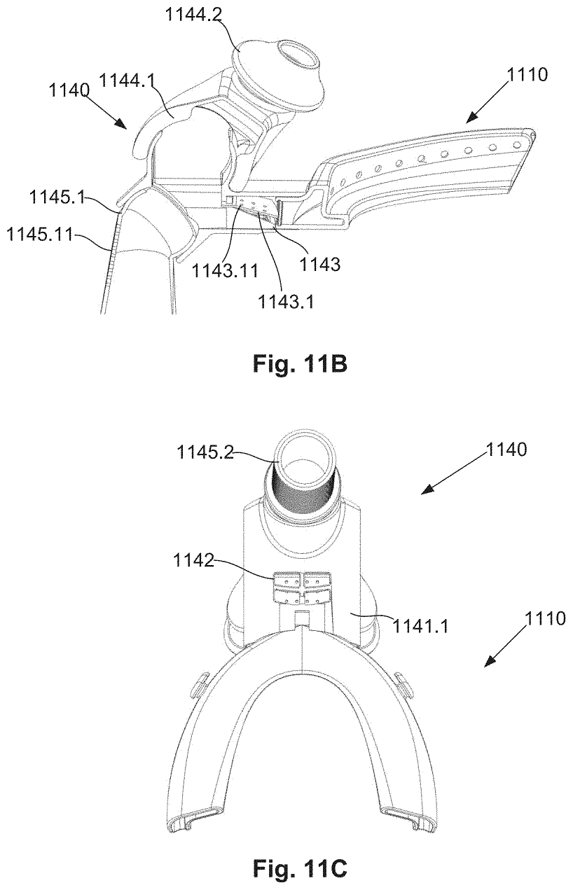

An alternative example will now be described with reference to FIGS. 11A to 11C. For the purpose of illustration similar features to those shown in FIGS. 8A to 8D are used to represent similar features, albeit increased in value by 300, and these will not be described in further detail.

In this example, the valve arrangement includes a hollow tube shaped valve body 1141.1 shaped to fit over the tubular body 731.1 of the extra-oral opening 731. The valve body 1141.1 also includes valve openings 1142 in an underside of the valve body 1141.1. A valve member 1143.1 is provided, mounted on a frame 1143, and which is movable between a first open position and a second closed position to at least partially close the openings 1142. The valve member 1143 also includes openings 1143.11 to allow airflow during exhalation and thereby provide differential resistance during inhalation and exhalation through the oral cavity.

In this example, the apparatus includes a nasal pillow 1144.2 attached to a nasal pillow connector body 1144.1, which is in turn attachable to the valve body 1141.1, via a pillow mounting 1141.4, which projects upwardly from the valve body 1141.1. In this example, the pillow mounting 1141.4 and nasal pillow connector body 1144.1 are shaped to form a cylindrical cup and socket connection, allowing rotation of the nasal pillow connector body 1144.1 so that an angle of the nasal pillow 1144.2 can be adjusted. Additionally and/or alternatively, the pillows could be mounted via ball joints, for additional adjustability.

The arrangement further includes a device connector 1145.1 coupled to a tube 1145.2 defining a device airway that supplies airflow from a positive airway pressure device. In this example, the device airway is in fluid communication with the nasal airway, allowing positive air pressure to be supplied to the nasal cavity. A number of device connector airway openings 1145.11 are provided to control air pressure in the device connector airway. Again the openings could be used in conjunction with a valve member to assist in controlling air pressure during inhalation and exhalation.

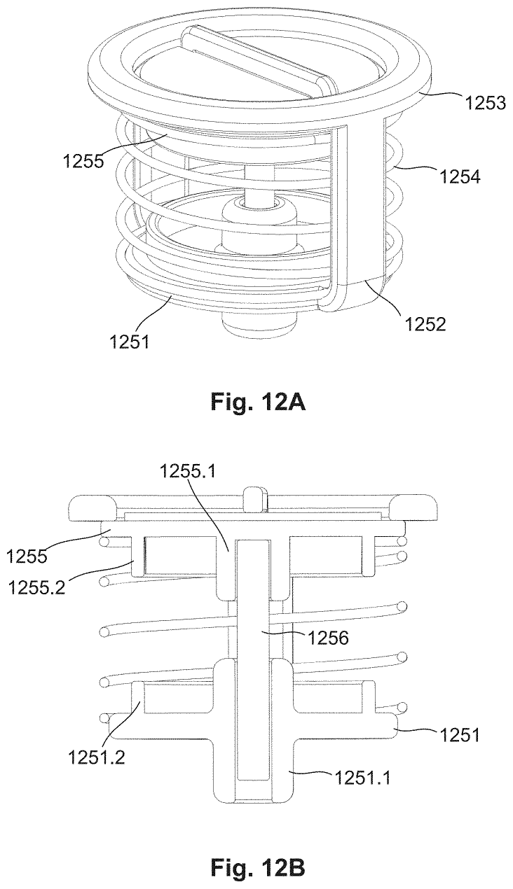

An example of an alternative valve arrangement will now be described with reference to FIGS. 12A and 12B. It will be appreciated that this can be incorporated into any of the valve bodies in the above described examples, and can be used instead of the flap type valve members.

In this example, the valve 1250 includes a disc shaped base 1251, spaced from a valve seat ring 1253, by longitudinally extending spaced parallel arms 1252. The base 1251 includes a central tube 1251.1, slidably supporting a shaft 1256, which is in turn mounted in a socket 1255.1 in a valve member 1255, allowing the valve member 1255 to slide longitudinally towards and away from the valve seat ring 1253. The base and valve member 1251, 1255 include upstanding rings 1251.2, 1255.2 on facing sides, which seat a spring 1254 extending from the base 1251 to the valve member 1255, thereby urging the valve member 1255 against the valve seat 1253 to close the valve. In this instance, the valve seat ring 1253 can be integrated into the side wall of a valve body, allowing the valve to open during inhalation and close when inhalation stops, thereby providing differential resistance during inhalation and exhalation as previously described.

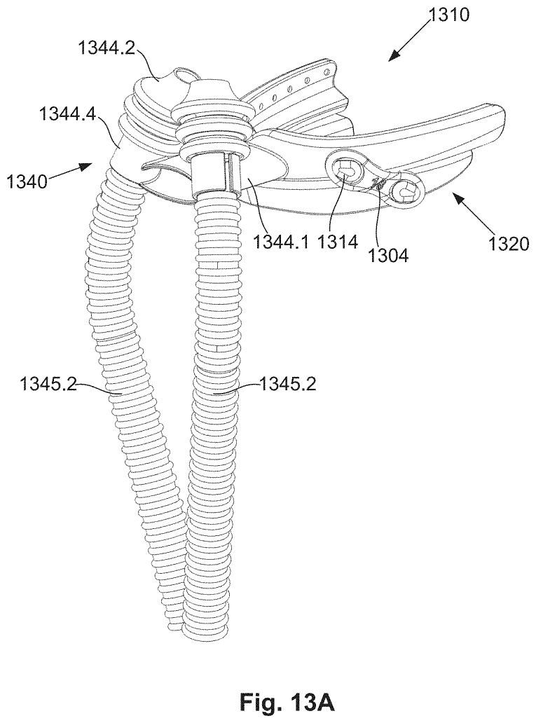

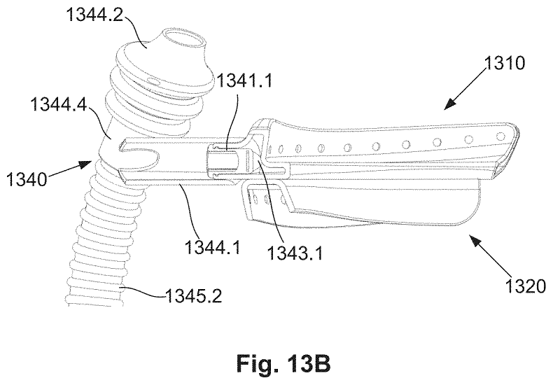

A further example arrangement will now be described with reference to FIGS. 13A and 13B. For the purpose of illustration similar features to those shown in FIGS. 8A to 8D are used to represent similar features, albeit increased in value by 500, and these will not be described in further detail.

In this example, the valve arrangement includes a hollow tube shaped valve body 1341.1 shaped to fit within the extra-oral opening of the first body 1310. The valve body 1341.1 is similar to that shown in FIGS. 4A to 4D, includes also includes valve members 1343.1 in a rear of the valve body 1341.1. As such an arrangement has been previously described, this will not be described in any further detail.

In this example, the apparatus includes a nasal pillow 1344.2 attached to a nasal pillow connector body 1344.1, which is a tubular body fitted over the extra-oral opening. The nasal pillows are fitted on the ends of device connector tubes 1345.2, which are coupled to the nasal pillow connector body 1344.1, via clips 1344.4 extending laterally from the nasal pillow connector body 1344.1.

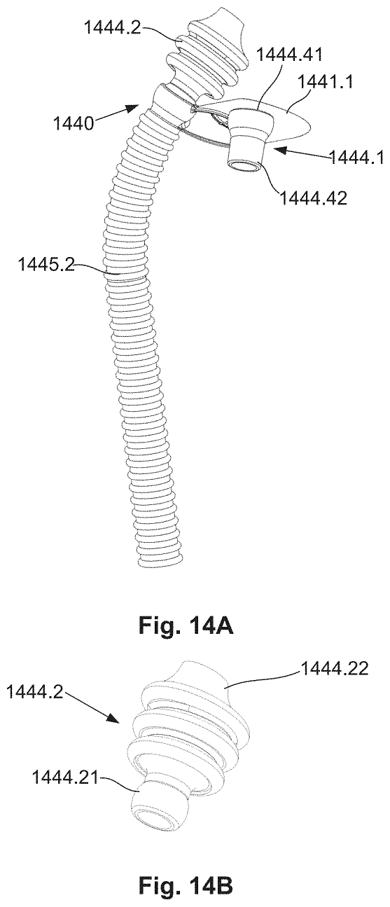

A further example arrangement will now be described with reference to FIGS. 14A and 14B. For the purpose of illustration similar features to those shown in FIGS. 13A and 13B are used to represent similar features, albeit increased in value by 100, and these will not be described in further detail.

In this example, the valve arrangement 1440 includes a hollow tube shaped valve body 1441.1 shaped to fit over or within the extra-oral opening of a first body (not shown). The valve body 1441.1 is similar to that shown in FIGS. 4A to 4D, includes also includes valve members in a rear of the valve body. As such an arrangement has been previously described, this will not be described in any further detail.

In this example, the apparatus includes nasal pillows 1444.2, each of which is attached to a respective connector tube 1444.1, mounted on the valve body 1441.1. The nasal pillows 1444.2 and connector tubes 1444.1 are coupled via a hollow ball and socket arrangement to allow air flow therethrough. The ball and socket arrangement includes a socket 1444.41 at a first upper end of the connector tubes 1444.1, and a ball 1444.21 at a first lower end of the nasal pillow 1444.2. The second upper end of the nasal pillow 1444.2 terminates in an opening 1444.22 that is inserted into the user's nasal passage, whilst the second lower end of the connector tube 1444.1 ends in a plug 1444.12 that can be inserted into an upper end of a device connector tube 1445.2, to allow connection to a positive air pressure device.

Accordingly, the above described arrangement allows each nasal pillow to be coupled to a respective connector tube via a hollow ball and socket arrangement, so that a relative orientation of each nasal pillow can be independently adjusted, thereby optimising the fit for different users. In this example, the ball of the ball and socket arrangement is provided on the nasal pillow, but it will be appreciated that this is not essential and alternatively the positions of the ball and socket could be reversed.

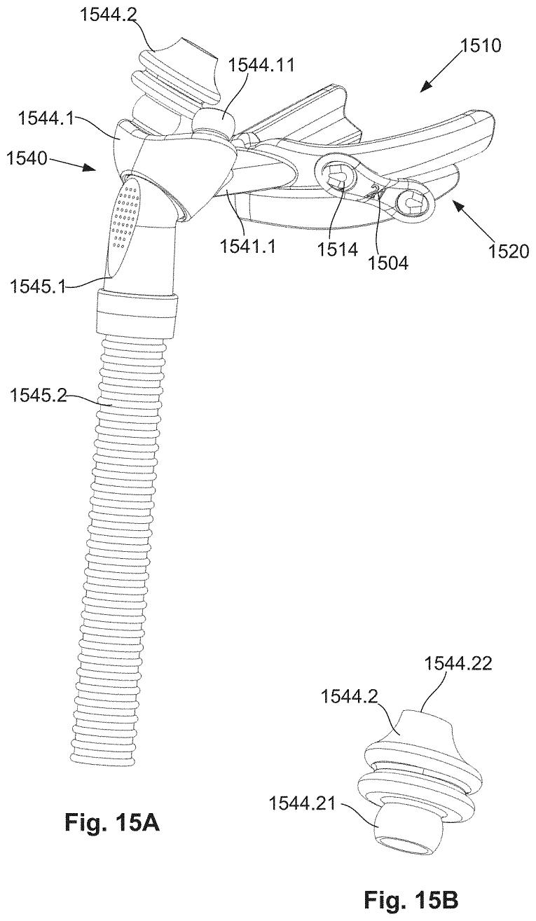

A further example arrangement will now be described with reference to FIGS. 15A and 15B. For the purpose of illustration similar features to those shown in FIGS. 11A to 11C are used to represent similar features, albeit increased in value by 400, and these will not be described in further detail.

In this example, the valve arrangement 1540 includes a hollow tube shaped valve body 1541.1 shaped to fit over or within the extra-oral opening of a first body 1510, which is attached to a second body 1520, via a connector 1504. The valve body 1541.1 is similar to that shown in FIGS. 11A to 11C, includes also includes valve members in the valve body. As such an arrangement has been previously described, this will not be described in any further detail.

In this example, the apparatus includes nasal pillows 1544.2, each of which is attached to a connector body 1544.1, mounted on the valve body 1541.1. The nasal pillows 1544.2 and connector body 1544.1 are coupled via a hollow ball and socket arrangement to allow airflow therethrough. The ball and socket arrangement includes two balls 1544.11 mounted on an upper end of the connector body 1544.1 and sockets 1544.21 at a lower end of the nasal pillows 1544.2. The second upper end of the nasal pillow 1544.2 terminates in an opening 1544.22 that is inserted into the user's nasal passage, whilst the second lower end of the connector body 1544.1 ends in a socket that receives a device connector 1545.1 coupled to a tube 1545.2 defining a device airway that supplies airflow from a positive airway pressure device.

Again this arrangement allows each nasal pillow to be coupled to a respective connector tube via a ball and socket arrangement, so that a relative orientation of each nasal pillow can be independently adjusted, thereby optimising the fit for different users. Again the position of the ball and socket could be reversed.

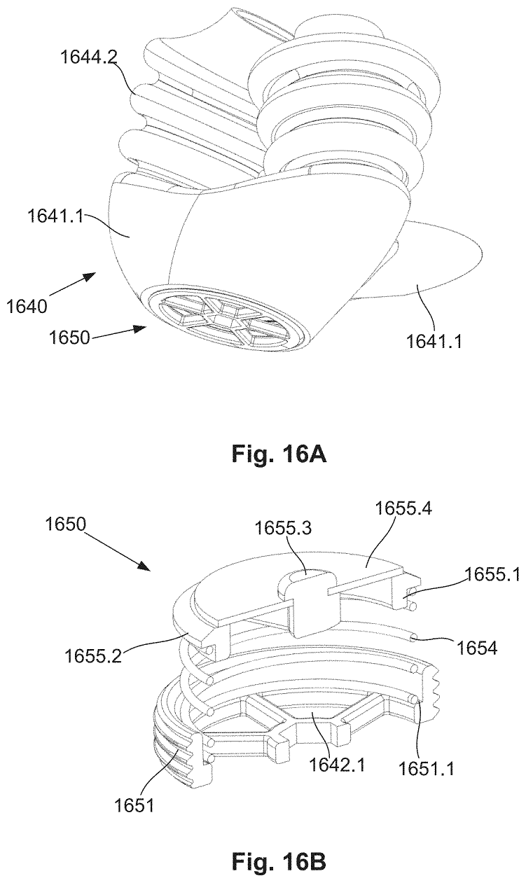

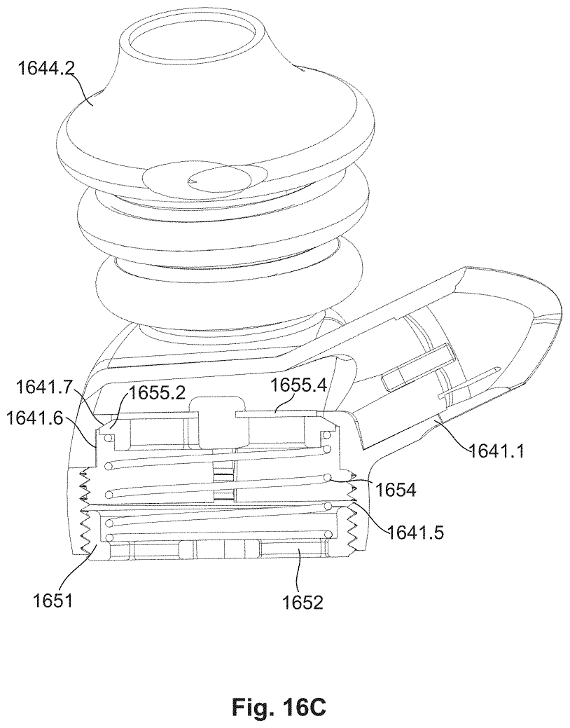

A further example arrangement will now be described with reference to FIGS. 16A to 16C.

In this example, the valve arrangement 1640 includes a hollow tube shaped valve body 1641.1 shaped to fit over or within the extra-oral opening of a first body (not shown). The valve body 1641.1 includes a valve body opening containing a valve 1650, to allow airflow into and out of the valve body to be controlled by the valve during inhalation and exhalation. In this example, the valve body 1641.1 also acts as a nasal pillow connector and supports nasal pillows 1644.2.

The valve is shown in more detail in FIG. 16B, and is broadly similar to the valve of FIGS. 12A and 12B, in that it includes a spring arrangement. In this instance, the valve 1650 includes a cylindrical base 1651, having an inwardly extending shoulder 1651.1 that acts as a seat for a first end of a spring 1654. The cylindrical base includes transverse struts 1652, defining a number of openings, to allow airflow into the valve. A second end of the spring 1654 is coupled to a valve ring 1655.1 positioned within the spring, and an outwardly extending sloped rim 1655.2, provided so that the spring engages an underside of the rim 1655.2. The valve ring 1655.1 supports a hub 1655.3, mounted on an arm extending radially inwardly from the valve ring 1655.1, with a flexible valve member 1655.4 mounted thereon. In use, the valve member 1655.4 selectively engages an upper end of the valve ring 1655.1 to thereby selectively close an opening defined by the valve ring 1655.1, depending on air flow therethrough.

In use, the valve 1650 is mounted in the valve body opening of the valve body 1641.1. This can be achieved using any suitable technique, such as frictional engagement, or the like. In one example, the cylindrical base 1651 has a threaded outer surface, which engages a correspondingly threaded inner surface 1641.5 of the valve body opening.

The inner surface of the valve body opening includes a narrow section 1641.6, that terminates in a sloped inwardly extending shoulder defining a valve seat 1641.7. In use, the spring 1654 urges the sloped rim 1655.2 of the valve ring 1655.1 into engagement with the valve seat 1655.1. During inhalation, airflow urges the valve member 1655.4 away from the valve ring 1655.1, allowing air to flow in through the openings 1652, and through the valve body, into the appliance airway. During exhalation, the valve member 1655.4 engages the valve ring 1655.2, and seals against the valve ring. Air pressure in the valve body 1641.1, pushes the valve ring 1655.1 downward, compressing the spring 1654 and disengaging the shoulder 1655.2 from the seat 1641.7, to allow outward airflow between the shoulder 1655.2 and seat 1641.7, through the openings 1652.

It will be appreciated that in this arrangement, the valve 1650 provides differential resistance during inhalation and exhalation using a combination of different mechanisms, integrated into a single common valve. In particular, this uses a flap type valve member 1655.4 to allow inhalation, with exhalation being controlled through the use of a spring valve, defined by interaction between the valve ring 1655.1 and the vale seat 1641.7.

Furthermore, the valve 1650 can be easily removed from the valve body 1641.1, allowing this to be easily replaced, so that different combinations of valve member properties and spring properties can be used to adjust the resistance during inhalation and exhalation, thereby matching the resistance to the particular requirements of the subject.

Accordingly, it will be appreciated that this arrangement provides a valve that is removably mounted to the valve body to allow valve members to be interchanged, and that similar functionality could also be achieved with other valves, such as those described above with respect to FIGS. 8A to 8D.

In this example, the valve arrangement includes a first valve to control resistance during inhalation and a second valve mechanism to provide resistance during exhalation. In particular, in this case, the valve member is mounted on a valve ring, and wherein the valve ring is biased into engagement with a valve seat within the valve body such that movement of the valve member controls resistance during inhalation and biasing of the valve ring against the valve seat controls resistance during exhalation.

In a preferred arrangement, the valve includes a base that in use is attached to the valve body, a spring and a valve ring, the valve ring supporting a valve member movable so that the valve ring is open during inhalation and closed during exhalation, and wherein the valve ring is biased into engagement with a valve seat so that the valve is open during exhalation and closed during inhalation.

Accordingly, it will be appreciated that the valve arrangement includes a single removable valve that uses different valve mechanisms that operate during inhalation and exhalation, respectively, to thereby provide differential resistance during inhalation and exhalation. Specifically, in one example, a movable valve member, such as a flap or similar, can be used to provide an airway during inhalation, whilst this closes during exhalation, to allow a spring valve to open and provide resistance during exhalation.

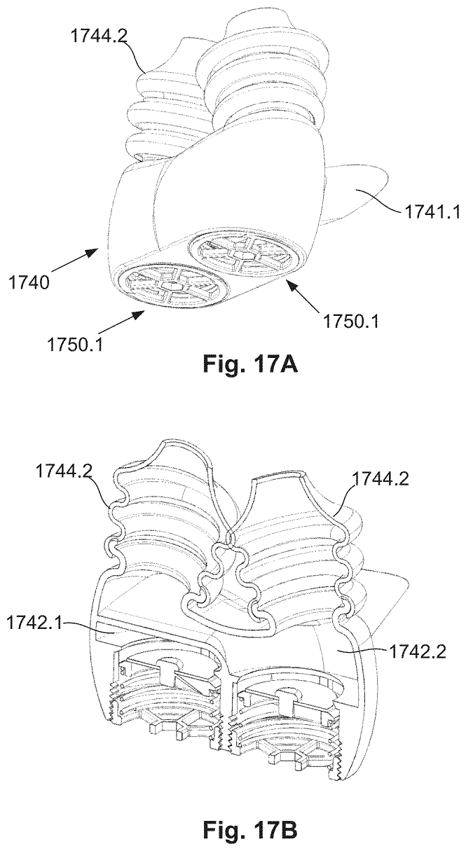

A further example arrangement will now be described with reference to FIGS. 17A and 17B. For the purpose of illustration similar reference numerals increased by 100 are used to refer to similar features to the previous example and such features will not be described in detail.

In this example, the valve arrangement 1740 includes a hollow tube-shaped valve body 1741.1 shaped to fit over or within the extra-oral opening of a first body (not shown). The valve body 1741.1 includes two valve body openings containing respective valve 1750.1, 1750.2, which have a similar form to the valve 1650 described above. In this example, the valve body 1741.1 also acts as a nasal pillow connector and supports nasal pillows 1744.2.

In this example, the body 1741.1 is divided internally to define oral and nasal airways 1742.1, 1742.2, in fluid communication with the oral appliance airway and nasal pillows respective. Each airway includes a respective valve arrangement 1750.1, 1750.2, to allow airflow into and out of the oral and nasal airways 1742.1, 1742.2 to be independently controlled by the respective valve 1750.1, 1750.2 during inhalation and exhalation. For example, inhalation could have a lower resistance via the nasal airway to encourage inhalation via the nose, whilst exhalation could have a lower resistance via the oral airway to maintain a positive pressure in the nasal airway.

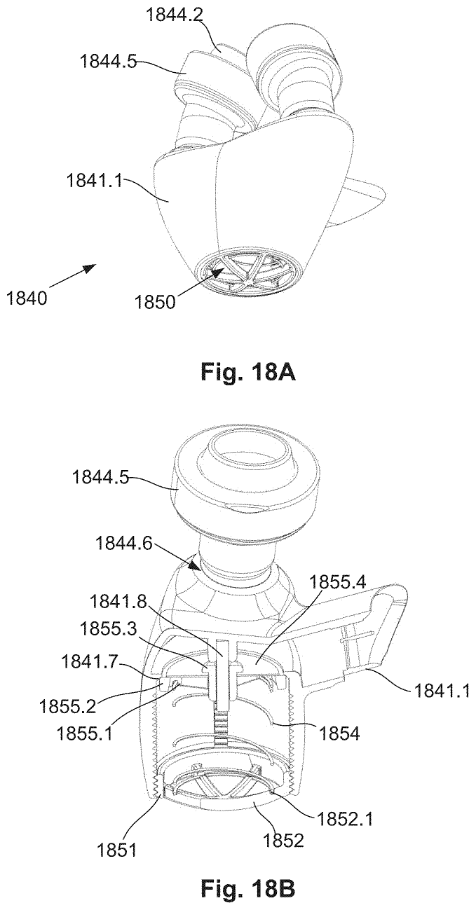

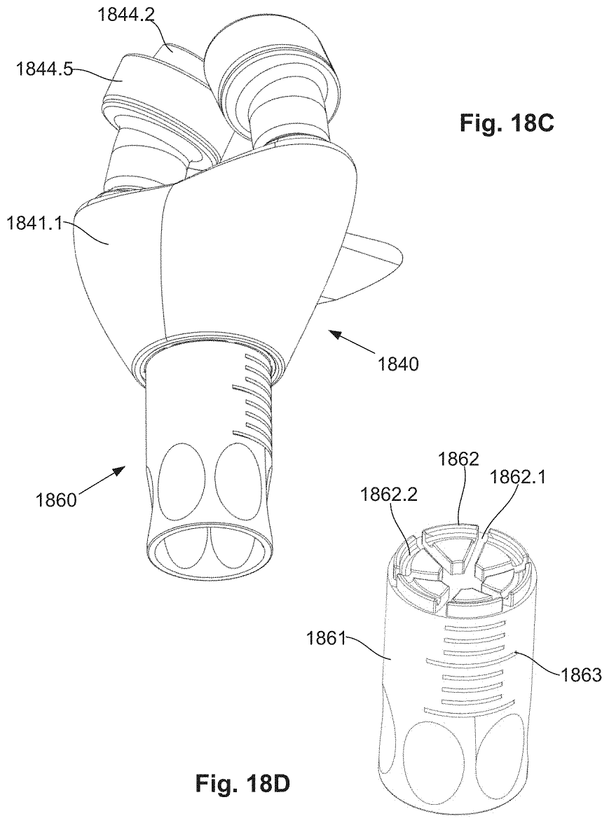

A further example arrangement will now be described with reference to FIGS. 18A to 18D.

In this example, the valve arrangement 1840 includes a hollow tube shaped valve body 1841.1 shaped to fit over or within the extra-oral opening of a first body (not shown). The valve body 1841.1 includes a valve body opening containing a valve 1850, to allow airflow into and out of the valve body to be controlled by the valve during inhalation and exhalation. In this example, the valve body 1841.1 also acts as a nasal pillow connector and supports nasal pillows 1844.2.

The valve is shown in more detail in FIG. 18B, and is broadly similar to the valve of FIG. 16B. In this instance, the valve 1850 includes a cylindrical valve base 1851, having radially extending transverse struts 1852 that defines openings to allow airflow into and out of the valve. The struts include a circumferential recess 1852.1 that acts as a seat for a first end of a spring 1854.

A second end of the spring 1854 is coupled to a valve ring 1855.1, having a shoulder 1855.2 that extends outwardly to define a 90° corner that engages a sloped valve seat 1841.7 within the valve body 1841.1. The valve ring 1855.1 supports a hub 1855.3, mounted on an arm extending radially inwardly from the valve ring 1855.1. The hub 1855.3 includes a cylindrical opening that in use is slidably engaged with a shaft 1841.8 extending downwardly within the valve body 1841.1, to thereby guide movement of the valve ring 1855.1 within the valve body 1841.1.

A flexible valve member 1855.4 is mounted on the valve hub 1855.3. In use, the valve member 1855.4 selectively engages an upper end of the valve ring 1855.1 to thereby selectively close an opening defined by the valve ring 1855.1, depending on air flow therethrough.

In use, the valve 1850 is mounted in the valve body opening of the valve body 1841.1. Whilst this can be achieved using any suitable technique, such as frictional engagement, or the like, in a preferred example, the cylindrical valve base 1851 has a threaded outer surface, which engages a correspondingly threaded inner surface 1841.5 of the valve body opening.

The inner surface of the valve body opening includes a narrow section 1841.6, which terminates in a sloped shoulder defining a valve seat 1841.7. In use, the spring 1854 urges the shoulder 1855.2 of the valve ring 1855.1 into engagement with the valve seat 1841.7. During inhalation, airflow urges the valve member 1855.4 away from the valve ring 1855.1, allowing air to flow in through the openings 1852, and through the valve body, into the appliance airway. During exhalation, the valve member 1855.4 engages the valve ring 1855.2, and seals against the valve ring. Air pressure in the valve body 1841.1, pushes the valve ring 1855.1 downward, compressing the spring 1854 and disengaging the shoulder 1855.2 from the seat 1841.7, to allow outward airflow between the shoulder 1855.2 and seat 1841.7, through the openings 1852.

It will be appreciated that in this arrangement, the valve 1850 provides differential resistance during inhalation and exhalation using a combination of different mechanisms, integrated into a single common valve. In particular, this uses a flap type valve member 1855.4 to allow inhalation, with exhalation being controlled through the use of a spring valve, defined by interaction between the valve ring 1855.1 and the vale seat 1841.7.

In the above arrangement, the degree of compression in the spring 1854 can control a degree of restriction during exhalation. Furthermore, the use of threaded engagement between the cylindrical valve base 1851 and the valve body 1841.1 can be used to adjust the position of the cylindrical valve base 1851 in the valve body 1841.1. This action adjusts a degree of compression in the spring, in turn controlling the restriction during exhalation.

To facilitate this process, an adjuster 1860 can be provided, which in one example is formed from a cylindrical adjuster body 1861 having projections 1862 on an upper end, which define radial and circumferential channels 1862.1, 1862.2. In use the channels 1862.1, 1862.2 receive the cylindrical valve base 1851 and radial struts 1852, so that rotation of the adjuster body 1861 allows the cylindrical valve base 1851 to be rotated and hence moved in and out within the valve body 1841.1. In a further example, the cylindrical adjuster body 1861 includes graduated markings 1863, allowing the position of the cylindrical valve base 1851 to be adjusted in a controlled manner. For example, movement of the cylindrical valve base 1851 by one marking can correspond to a set increase or decrease in exhalation resistance.

In addition to the above described modifications to the valve arrangement, in the above example, nasal pillows 1844.2 are provided, which are formed from rigid or semi-rigid pillow tubes 1844.2, made from nylon or polyurethane. The pillow tubes that are adjustably mounted to the valve body 1844.1 via a ball and socket mounting 1844.6, to allow an orientation of the tubes to be altered, whilst different lengths of tubes can be used to accommodate different mouth to nose heights. Gel inserts 1844.5 are mounted on the tubes to allow for sealing.

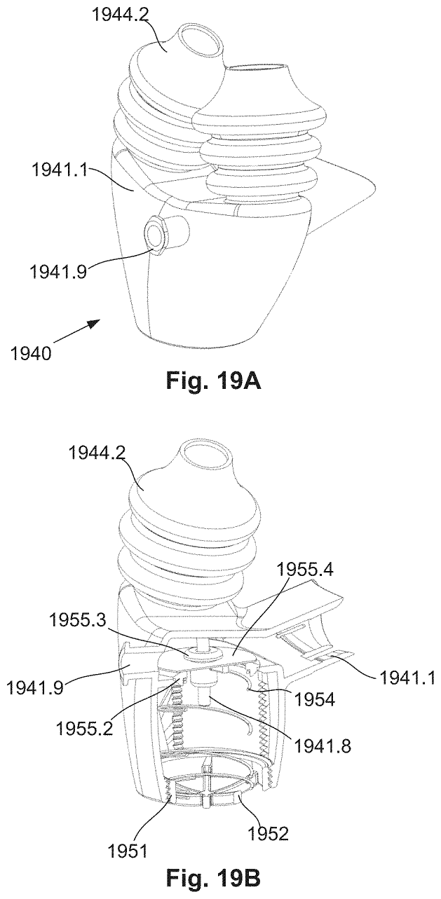

A further example arrangement will now be described with reference to FIGS. 19A and 19B, which show a modified version of the arrangement in FIGS. 18A to 18D. For ease of explanation, this explanation uses reference numerals similar to those used in the example of FIGS. 18A to 18D, albeit increased by 100, and such features will not therefore be described in further detail.

Accordingly, in this example, the valve arrangement 1940 includes a hollow tube-shaped valve body 1941.1 incorporating a valve 1950. In this example, nasal pillows 1944.2 similar to those previously described are provided.

In this example, the valve body 1941.1 further includes a port 1941.9, upstream of the valve 1950 and in fluid communication with the valve airway. In one example, the port 1941.9 is in the form of a tapered Luer fitting, allowing a hose or other delivery device to be coupled directly thereto. This allows for the delivery of gases, such as O₂, or the like, medications, anaesthetics, or other substances, directly into the valve airway. The valve arrangements allow airflow into an out of the user's airway, so that such medications and/or gases are entrained in the airflow and delivered to the user airway. This can also be used to deliver air under pressure to thereby provide PAP. In this example, continuous small vent holes are not required as is the case with regular PAP as pressure can be controlled using the valve arrangement, allowing the air volume, and hence pump size, to be reduced substantially. For example, PAP can be supplied at say 10 cmH₂O pressure. The nasal PEEP (Positive End Expiratory Pressure) provided by the valve arrangement can be set at say 12cmH₂O which will only open on exhalation as it is higher than the PAP pressure.

In one example, an arrangement of this form can be used in conjunction with a dual valve device similar to that described above with respect to FIGS. 17A and 17B, allowing a combination of PAP and PEEP to provided. As an example, in the dual configuration, nasal PAP can be supplied at say 10cmH₂O pressure. The nasal PEEP can be set at say 12cmH₂O which will only open on exhalation as it is higher than the PAP pressure. The oral PEEP can be set even higher.

In examples in which PAP is supplied via a port, a further one-way flapper valve may be required on the air supply hose for safety. Specifically, this can be used to prevent rebreathing into the hose and allows the arrangement to revert to a standard PAP or TwoPAP arrangement if the air supply fails.

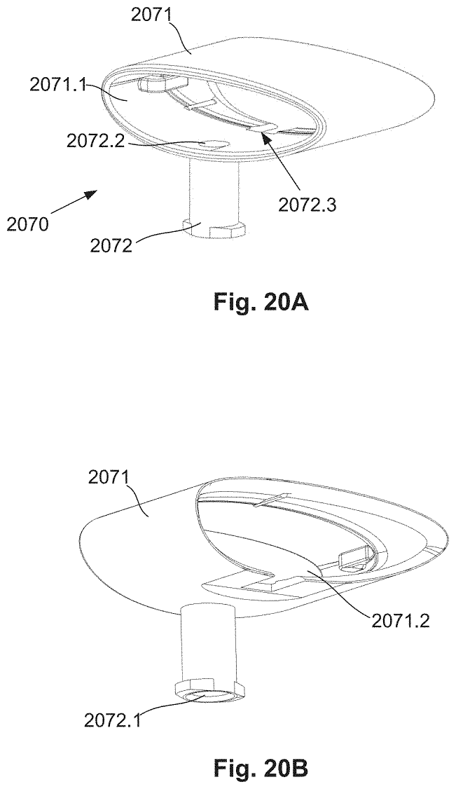

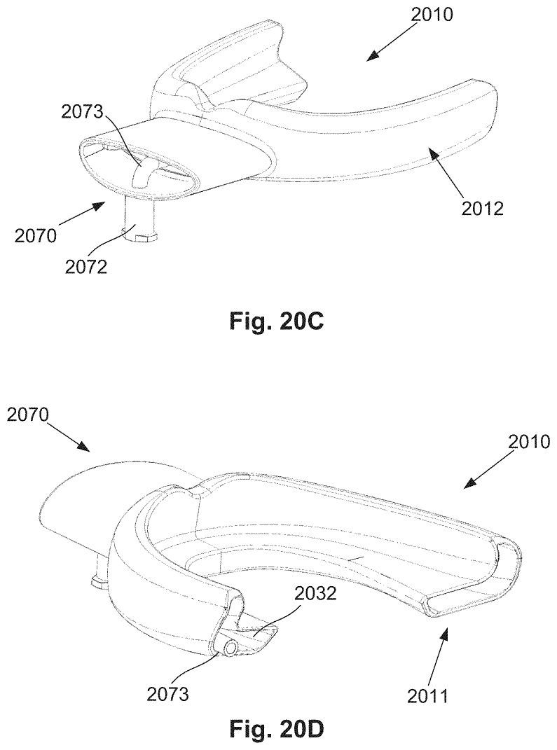

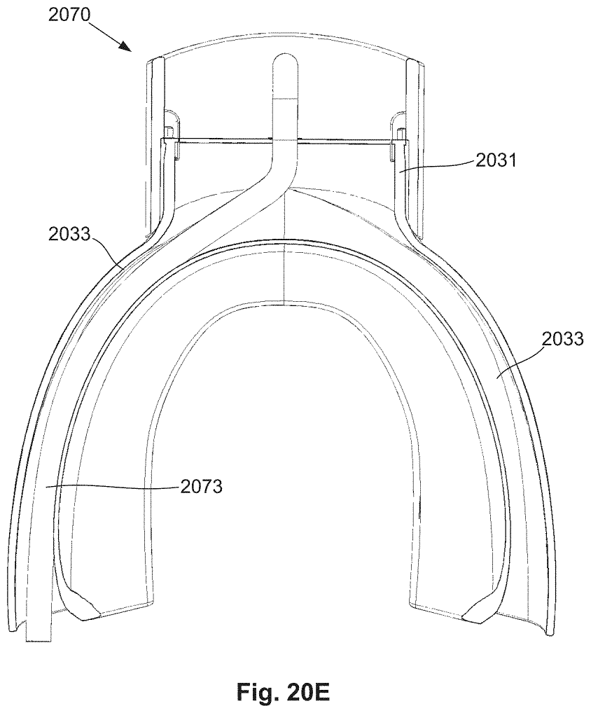

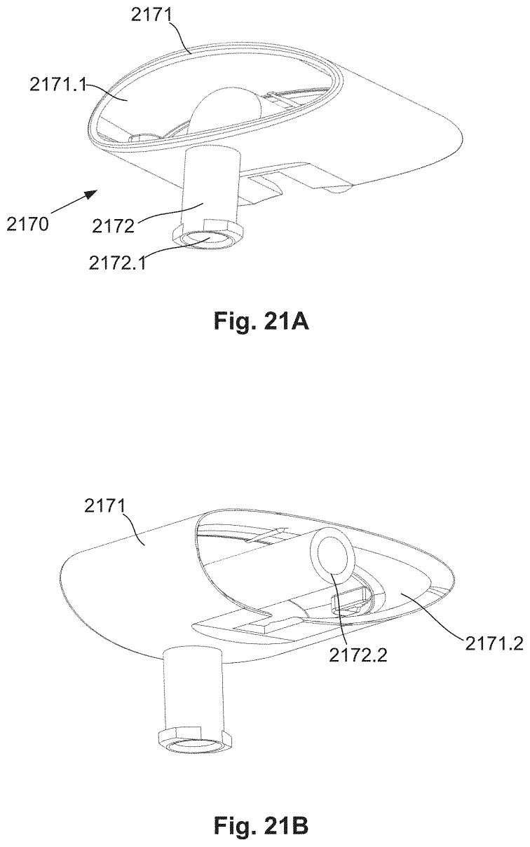

Such arrangements to provide air and or other gas flow can also be implemented without the valve arrangement and examples of this will now be described with reference to FIGS. 20A to 20E, and 21A and 21B.

In the example of FIGS. 20A and 20B, the connector arrangement 2070 includes hollow tube-shaped tubular connector body 2071 shaped to fit over or within the extra-oral opening 131 of a breathing assistance apparatus similar to that described above with respect to FIGS. 1A to 1E. The tubular body 2071 includes front and rear openings 2071.1, 2071.2 connected by a connector airway 2071.3, allowing airflow therethrough, enabling airflow into and out of the breathing assistance apparatus, and hence the user's airway.

Additionally, the connector includes a port 2072, similar to a Luer port, including an external opening 2072.1 and an internal opening 2072.2 in fluid communication with the connector airway 2071.3.

As shown in FIGS. 20C to 20E, the connector can be attached to an oral appliance similar to that described above with respect to FIGS. 1A to 1E. Accordingly, the oral appliance includes an appliance body 2010 shaped to be at least partially positioned within the oral cavity of a user. Specifically, in this example, the oral appliance includes a hollow lateral base 2011, extending inwardly from a hollow arcuate side wall 2012. The appliance body includes a tubular body 2131.1 that extends between lips of the user.

In this instance, the port 2072 is attached to a delivery tube, which extends through the connector body 2070 and the appliance airway 2033, terminating adjacent an intra-oral opening 2032. This allows gas, such as air, O₂, or the like to be delivered directly into the rear of the oral cavity.

It will also be appreciated that similar functionality could be achieved without requiring the port, for example, by positioning and retaining a delivery tube in the apparatus of FIGS. 1A to 1E, using clips or another similar arrangement.

In the example of FIGS. 20A and 20B, the internal opening 2072.2 terminates at an internal wall of the tubular body 2071. In contrast in the alternative configuration of FIGS. 21A and 21B, the port 2172 extends through the tubular body airway 2171.3 so that the port internal opening 2172.2 is proximate the rear opening 2171.2.

These arrangements again enable delivery of gases, such as O₂, air, or the like, medications, anaesthetics, or other substances, directly into the valve airway. The valve arrangements allow airflow into an out of the user airway, so that such medications and/or gases are entrained in the airflow and delivered to the user airway.

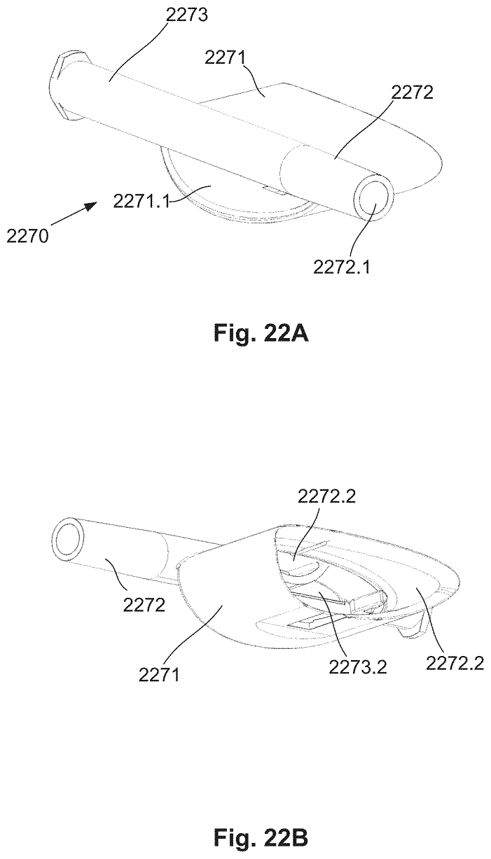

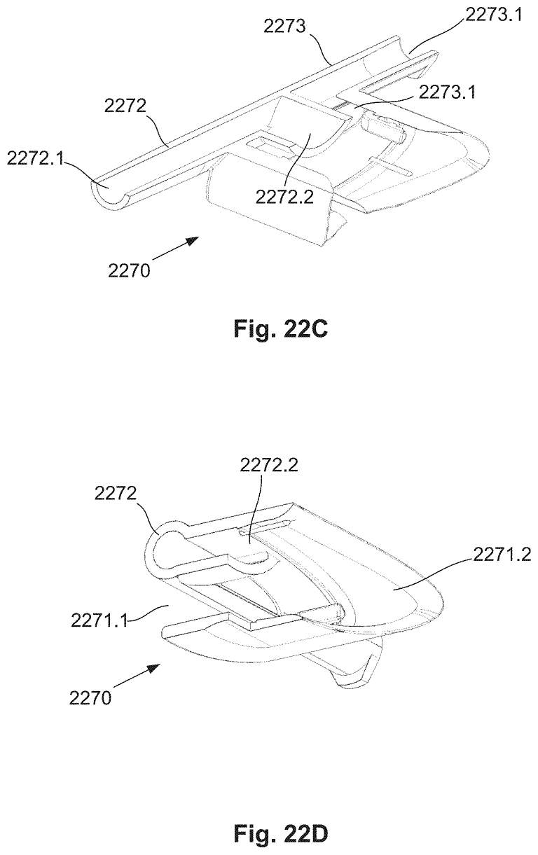

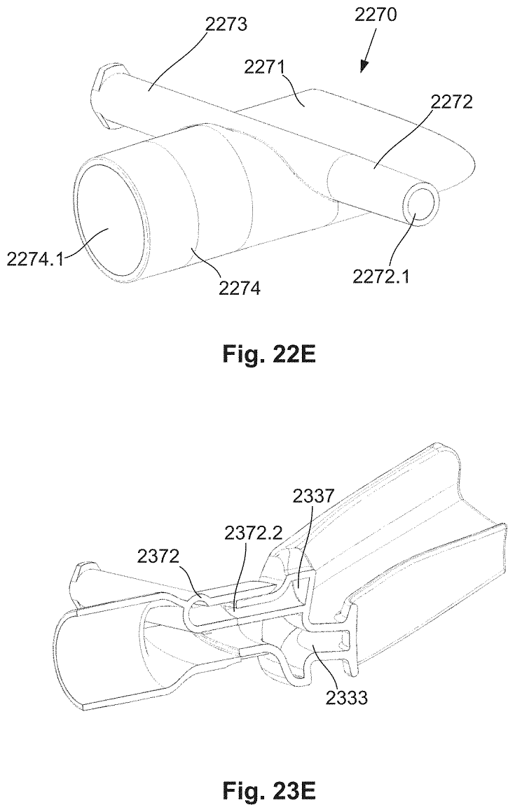

A further example connector arrangement will now be described with reference to FIGS. 22A to 22D.

In this example, the connector arrangement 2270 includes hollow tube-shaped tubular connector body 2271 shaped to fit over or within the extra-oral opening of an oral appliance. The tubular body 2271 includes front and rear openings 2271.1, 2271.2 connected by a connector airway 2071.3, allowing airflow therethrough, enabling airflow into and out of the oral appliance, and hence the user's airway.

Additionally, the connector includes a first port 2272, extending laterally from an upper portion of the connector body 2271, including an external opening 2272.1 and an internal opening 2272.2. The connector further includes a second port 2273, extending laterally from the connector body 2271, and aligned in opposition to the first port 2272. The second port includes an external opening 2273.1 and an internal opening 2273.2, and is not in fluid communication with the first port to define two distinct flow paths. In use the first and second ports can be provided to allow different gases to be supplied to or received from the user's oral cavity, with this typically being achieved via an intra-oral opening, and optionally via separate intra-oral openings, via respective appliance airways. This can be used to allow O₂, anaesthetics, or other gases to be supplied, typically via the appliance airway, for delivery to the user. Meanwhile the second port can be used to collect exhaled gases, for example including Luer lock for the purpose of measuring end tidal volume.

The connector can also be coupled to a front port 2174, having an external opening 2174.1, and internal opening (not shown), in fluid communication with the front opening 2271.1, which can be used for connection to further equipment, such as an inline valve, which can be used to provide PEEP, as will be described in more detail below, or for the attachment of respiratory equipment, such as positive air pressure devices, Ambu bag for resuscitation, or the like.

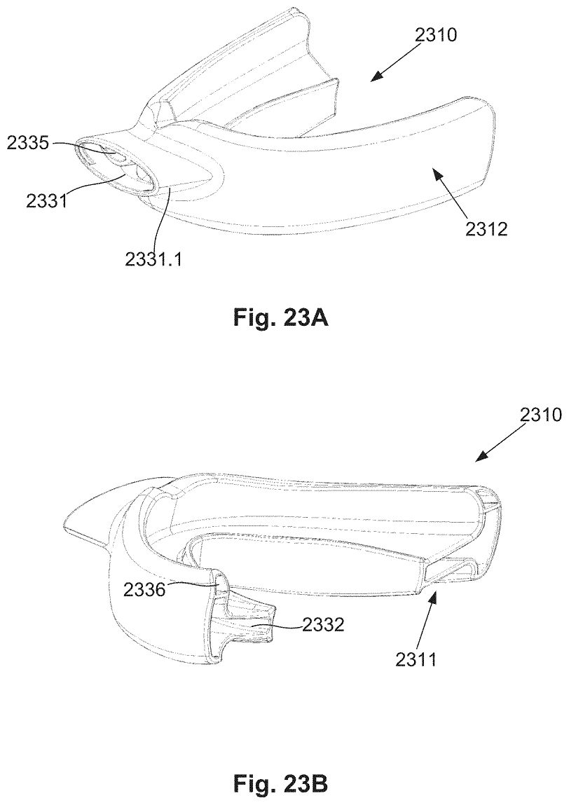

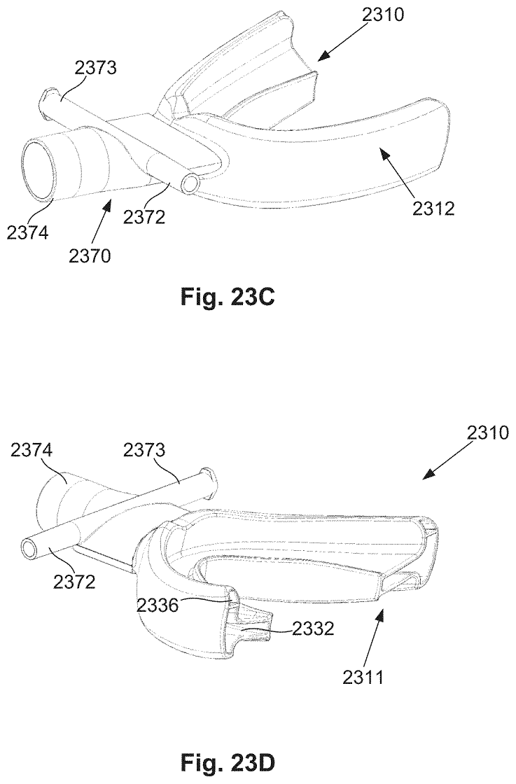

In one example, each port 2272, 2273 can be provided in fluid communication with a common appliance airway, but in another example, respective airways can be provided, and an example of this will now be described with reference to FIGS. 23A to 23E.

In this example, an oral appliance is provided having a single appliance body 2310 shaped to be at least partially positioned within the oral cavity of a user. Specifically, in this example, the oral appliance includes a hollow lateral base 2311, extending inwardly from a hollow arcuate side wall 2312. The appliance body includes a tubular body 2331.1 that extends between lips of the user.

In this instance, the tubular body 2331.1 includes a first extra-oral inlet 2335 provided in communication with first intra-oral openings 2336 via first channels 2337 and a second extra-oral opening 2331 in fluid communication with second intra-oral openings 2332, via a second channel 2333. In this example, the first inlet 2335 is configured to connect with the first port internal opening, so that it is in fluid and thereby be in fluid communication with the first port 2372, allowing O₂, or the like to be delivered directly into the rear of the oral cavity via the first intra-oral openings 2336, whilst the second port 2373 is in fluid communication with the second channel 2333, allowing exhaled air to be collected for the purpose of determining the end tidal volume.

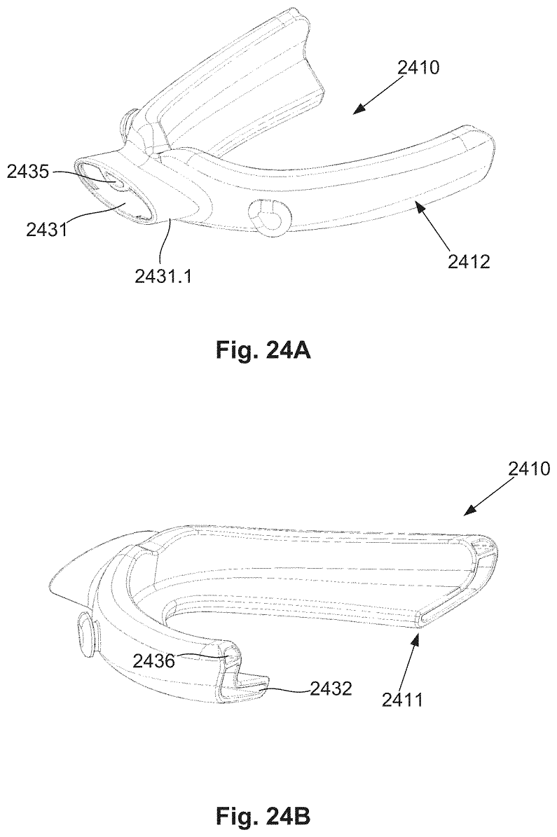

It will be appreciated that the example of FIGS. 23A to 23E uses a single body oral appliance capable of receiving maxillary and mandibular teeth, but this is not essential, and it will be appreciated that a similar configuration can be implemented with a dual body oral appliance, similar to that shown in FIGS. 7A and 7B, and an example of this will now be described in more detail with reference to FIGS. 24A and 24B.

In this example, the oral appliance body 2410 is shaped to be at least partially positioned within the oral cavity of a user and engage the maxillary teeth. The oral appliance includes a hollow lateral base 2411, extending inwardly from a hollow arcuate side wall 2412, with a tubular body 2331.1 extending forwardly so as to extend between lips of the user in use.

The tubular body 2431.1 again includes a second extra-oral opening 2431 in fluid communication with second intra-oral openings 2432, via second channels, and a first extra-oral opening 2435 in fluid communication with first intra-oral openings 2436 via first channels, allowing the first and second ports to be provided in fluid communication with the first and second intra-oral openings 2432, 2436, respectively.

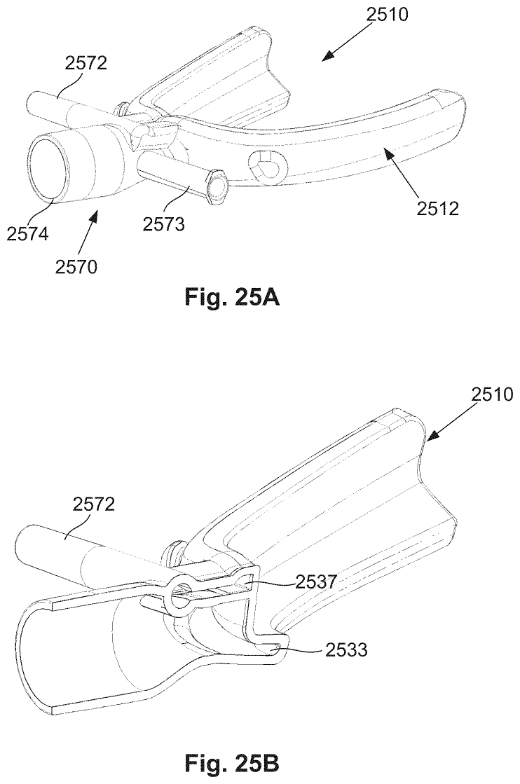

An alternative connector arrangement used with the oral appliance of FIGS. 24A and 24B is shown in FIGS. 25A and 25B.

In this example, the oral appliance includes a body 2510 similar to the body 2410 described above. In this example, the connector 2570 includes a first port 2572 in fluid communication with a first airway 2537. A second port 2573 is also provided in fluid communication with a second airway 2533, and it will therefore be appreciated that operation is substantially similar to that described above. In this example however, the first and second ports 2572, 2573 are vertically offset, which can facilitate connectivity with the respective airways.

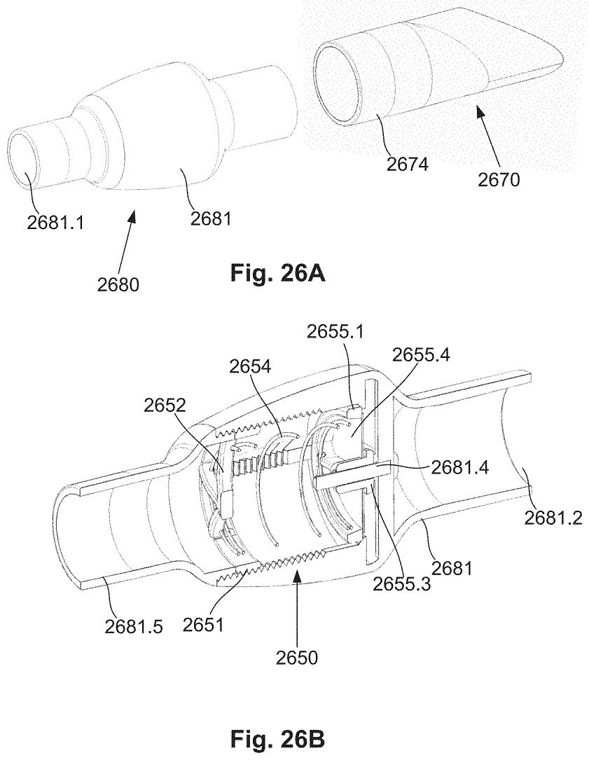

In one example, the above arrangements can be used with an inline valve arrangement, and example of which will now be described with reference to FIGS. 26A and 26B.

In this example, the valve arrangement 2680 includes a valve body 2681, including front and rear openings 2681.1, 2681.2. A valve arrangement 2650 is provided in the body, which is generally similar to the valve arrangement described with respect to FIGS. 18A to 18D.

Specifically, in this arrangement, the 2650 includes a cylindrical valve base 2651, having radially extending transverse struts 2652 that defines openings to allow airflow into and out of the valve. The struts include a circumferential recess that acts as a seat for a first end of a spring 2654. A second end of the spring 2654 is coupled to a valve ring 2655.1, having a shoulder that extends outwardly to define a 90° corner that engages a valve seat within the valve body 2681. The valve ring 2655.1 supports a hub 2655.3, mounted on an arm extending radially inwardly from the valve ring 2655.1. The hub 2655.3 includes a cylindrical opening that in use is slidably engaged with a shaft 2681.4 extending axially within the valve body 2681, to thereby guide movement of the valve ring 2655.1 within the valve body 2681.

A flexible valve member 2655.4 is mounted on the valve hub 2655.3. In use, the valve member 2655.4 selectively engages an upper end of the valve ring 2655.1 to thereby selectively close an opening defined by the valve ring 2655.1, depending on air flow therethrough.

In use, the valve 2650 is mounted in the valve body 2681 via a threaded outer surface of the cylindrical valve base 2651, which engages a correspondingly threaded inner surface of the valve body 2681, and which may be held in place via a front opening body 2681.5.

It will be appreciated that in this arrangement, the valve 2650 provides differential resistance during inhalation and exhalation using a combination of different mechanisms, integrated into a single common valve. In particular, this uses a flap type valve member 2655.4 which opens during inhalation to provide a first level of resistance, with exhalation being controlled through the use of a spring valve, defined by interaction between the valve ring 2655.1 and the vale seat 2641.7.

In the above arrangement, the degree of compression in the spring 2654 can control a degree of restriction during exhalation. Furthermore, the use of threaded engagement between the cylindrical valve base 2651 and the valve body 2681 can be used to adjust the position of the cylindrical valve base 2651 in the valve body 2681. This action adjusts a degree of compression in the spring, in turn controlling the restriction during exhalation.

It will be appreciated that the inline valve arrangement can be used in conjunction with the front connector port 2674, allowing the inline valve to be used with the port arrangements of FIGS. 22 to 25.

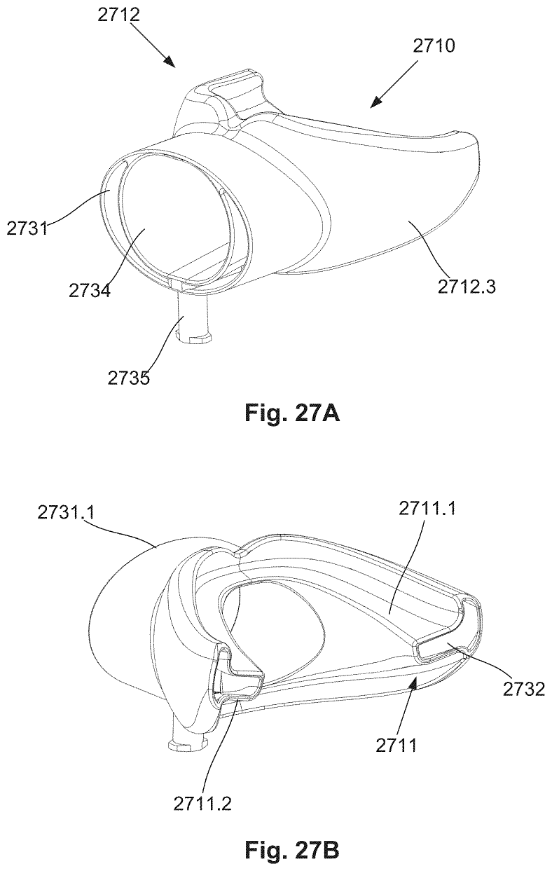

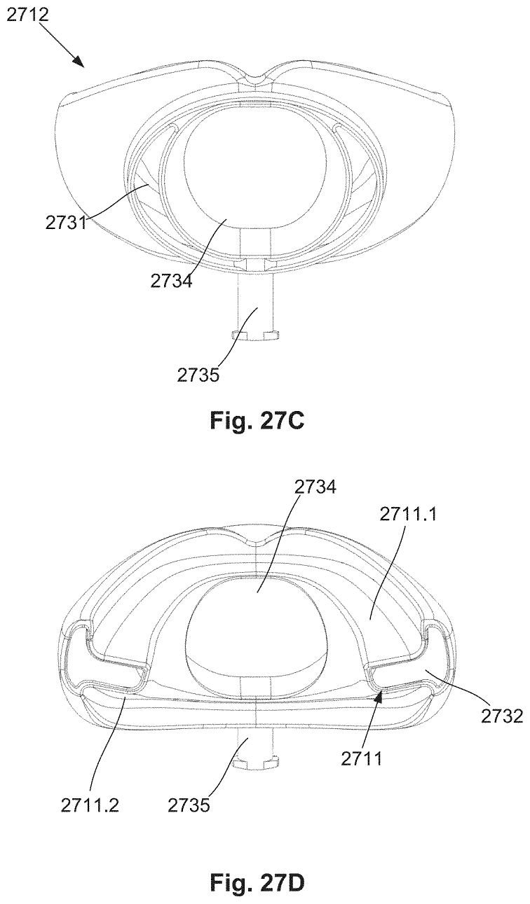

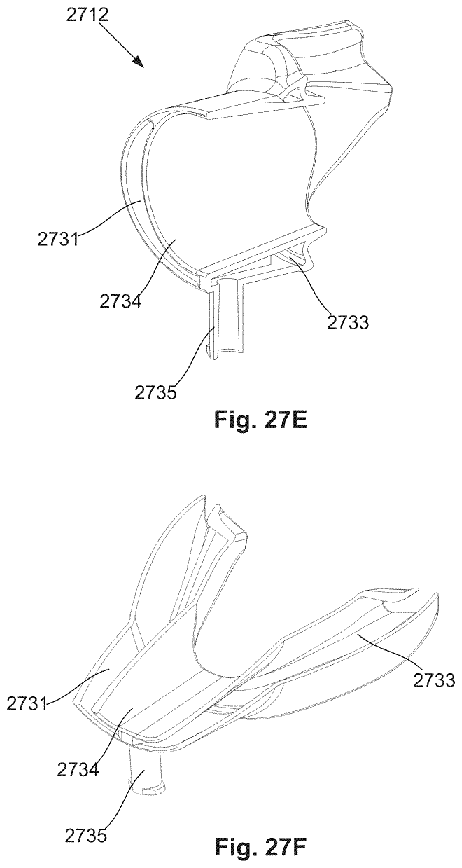

A further example of a breathing assistance apparatus will now be described with reference to FIGS. 27A to 27F.

The oral appliance includes an appliance body 2710 shaped to be at least partially positioned within the oral cavity of a user. Specifically, in this example, the oral appliance includes a hollow lateral base 2711, having spaced upper and lower surfaces 2721.1, 2721.2, extending inwardly from a hollow arcuate side wall 2712, having upper and lower inner side walls spaced from a curved outer side wall 2712.3. The appliance body includes a tubular body 2731.1 that extends between lips of the user.

In this example, an extra-oral opening 2731 is provided on outer sides of the tubular body 2731.1 with the extra-oral opening 2731 being in fluid communication with an appliance airway 2733 passing through the body 2710, to one or more intraoral openings 2732 provided in the oral cavity, to allow airflow into and out of a posterior region of the oral cavity.

A port 2735 is provided which extends from the tubular body airway 2731.3, and is in fluid communication with the appliance airway 2733 to enable delivery of gases, such as O₂, air, or the like, medications, anaesthetics, or other substances, directly into the oral appliance airway.

Additionally, in this example, a second extra-oral opening 2734 is provided, that is within the tubular body 2731.1 and in inwardly of the extra-oral opening 2731, which opens directly into the front of the user's oral cavity. This allows access to the user's extra-oral cavity, for example for insertion of tools, or equipment, or similar.

This arrangement is particularly suited for use in surgical applications, as it allows delivery of medication, such as anaesthetics via the port 2735, and to allow access to the oral cavity via the opening 2734, for example to allow surgical instruments, such as gastroscopy/endoscopy instruments to be received and inserted into the oral cavity.

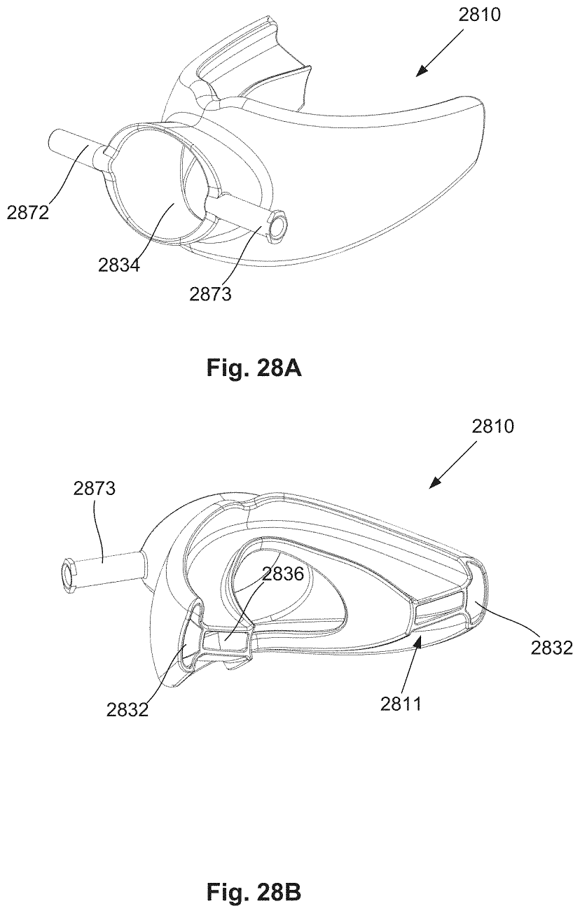

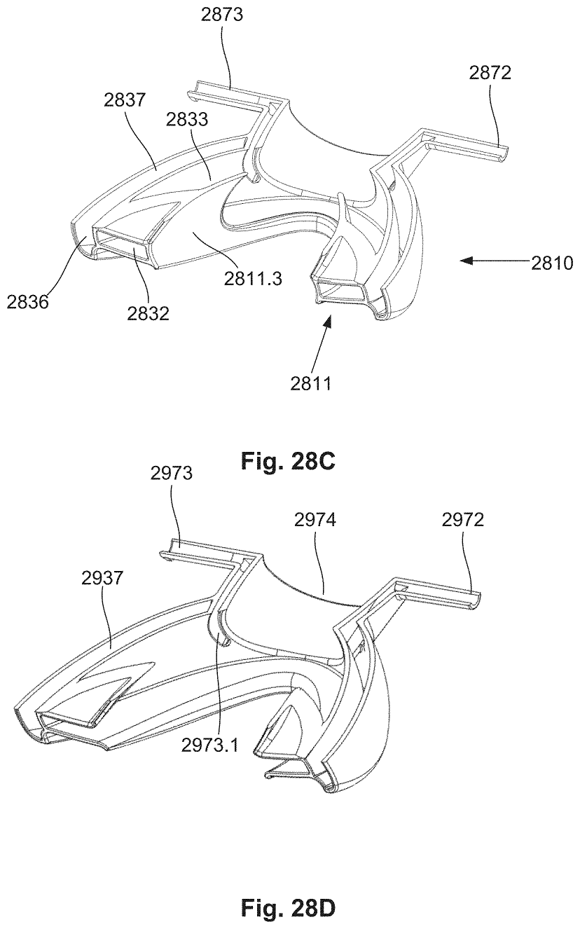

A further variation is shown in FIGS. 28A to 28C.

In this example, the opening 2371 is replaced by two ports 2872, 2873, which communicate via respective primary and second airways 2837, 2833, with primary and secondary intra-oral openings 2836, 2832. In this instance, the second airway 2833 is partially enclosed by a lingual wall 2811.3, but in the variation shown in FIG. 28D, the lingual wall is removed, so that second port 2873 opens directly into the oral cavity via an opening 2873.1. In another case, O₂, or other gases to be delivered via the port 2872, with end tidal volume being measured via port 2873. As in the previous example, access to the oral cavity can be provided via an opening 2874.

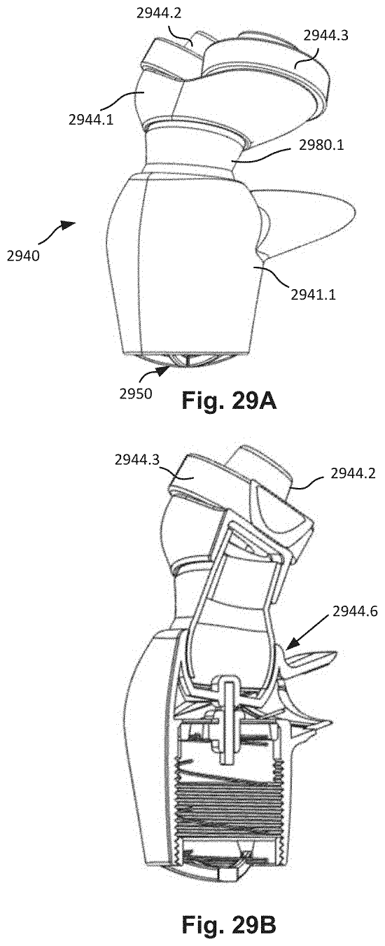

A further example of a valve arrangement will now be described with reference to FIGS. 29A and 29B, which shows a modified version of the valve arrangement shown in FIGS. 18A to 18D. For ease of explanation, this explanation uses reference numerals similar to those used in the example of FIGS. 18A to 18D, albeit increased by 1100, and such features will not therefore be described in further detail.

Accordingly, in this example, the valve arrangement 2940 includes a hollow tube shaped valve body 2941.1 shaped to fit over or within the extra-oral opening of a first body (not shown). The valve body 2941.1 includes a valve body opening containing a valve 2950, to allow airflow into and out of the valve body to be controlled by the valve during inhalation and exhalation. The valve 2950 shares the same configuration as the valve 1850 described above with reference to FIGS. 18A to 18D, and thus will not be described in further detail. It is arranged so as to be in fluid communication with both the nasal pillow tubes 2944.2 of the valve arrangement as well as the extra-oral opening when fitted to the first body.

In this example, the valve arrangement 2940 also includes a nasal pillow connector 2944.1 mounted to the valve body 2941.1 via a hollow nasal pillow connector stalk 2980.1. The nasal pillow tubes 2944.2 are formed integrally with the nasal pillow connector 2944.1, from a rigid or semi-rigid material such as nylon, polyurethane or silicone rubber. The nasal pillow connector stalk 2980.1 is adjustably mounted to the valve body 2941.1 via a hollow ball and socket mounting 2944.6, to allow an orientation of the nasal pillows 2944.2 to be altered. Gel rings 2944.3 are mounted on the nasal pillow tubes 2944.2.

The nasal pillow connector 2944.1 can be coupled to the nasal pillow connector stalk 2980.1 in a variety of manners. In this example, the nasal pillow connector 2944.1 is attached to the nasal pillow connector stalk 2980.1 in a reversible manner, to allow the nasal pillow connector 2944.1 to be attached to and then subsequently removed from the nasal pillow connector stalk 2980.1. Such reversible coupling arrangements can include magnetic engagement, the use of an interference fit, a clip fit or, as is the case in this example, a friction fit.

Such reversible coupling arrangements allow the nasal pillow connector 2944.1 to be interchanged. Accordingly, different mouth to nose heights and different nostril opening sizes and orientations can be readily accommodated by attaching an appropriately configured nasal pillow connector 2944.1. With such a modular design, the various aspects that can be changed between different nasal pillow connectors 2944.1 can include: the angle at which the tubes of the nasal pillows 2944.2 project; the length and/or diameter of the tubes of the nasal pillows 2944.2; the shape of the tubes of the nasal pillows 2944.2 (e.g. substantially circular or substantially elliptical); the overall height and/or width of the connector, etc. The gel rings 2944.3 may then improve sealing between the nasal pillows and the nostrils of a user by compensating for any minor discrepancies in fit between the closest fitting nasal pillow connector and the attributes of the user's face.

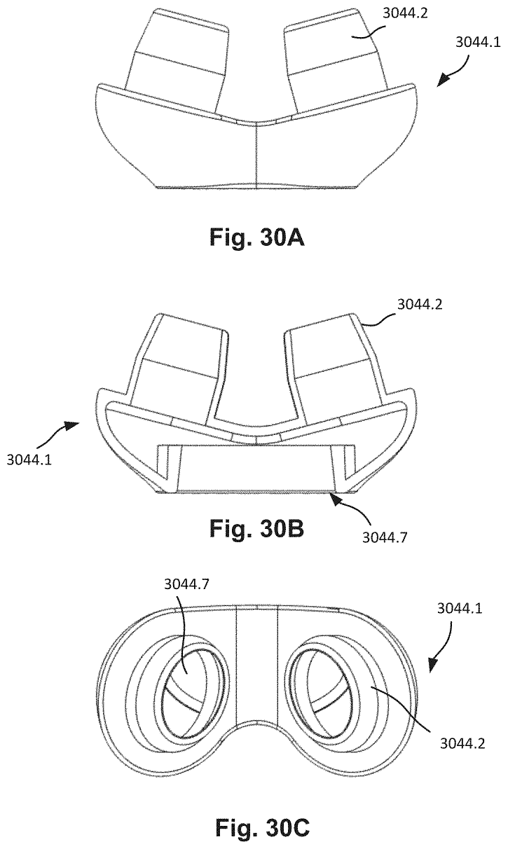

Another example of an interchangeable nasal pillow connector 3044.1 integrally formed with nasal pillow tubes 3044.2 is shown in FIGS. 30A to 30C. A lower surface of the nasal pillow connector 3044.1 defines a substantially elliptical lower opening 3044.7 in fluid communication with the nasal pillow tubes 3044.2 and configured to be coupled to a nasal pillow connector stalk with a friction fit.

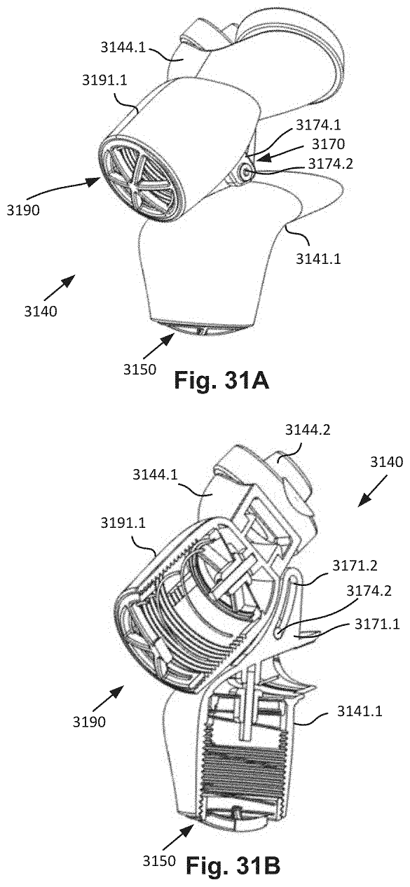

A further example of a valve arrangement will now be described with reference to FIGS. 31A and 31B, which shows a modified version of the valve arrangement shown in FIGS. 29A and 29B. For ease of explanation, this explanation uses reference numerals similar to those used in the example of FIGS. 29A and 29B, albeit increased by 200, and such features will not therefore be described in further detail.

Accordingly, in this example, the valve arrangement 3140 includes a hollow tube shaped oral valve body 3141.1 shaped to fit over or within the extra-oral opening of a first body (not shown). In addition, in this example, the valve arrangement 3140 also includes a hollow tube shaped nasal valve body 3191.1 mounted to the oral valve body 3141.1 via a sliding adjustment arrangement 3170. The oral valve body 3141.1 includes a valve body opening containing an oral valve 3150, to allow airflow into and out of the oral valve body 3141.1 to be controlled by the oral valve 3150 during inhalation and exhalation through a user's oral airway. The nasal valve body 3191.1 also includes a valve body opening containing a nasal valve 3190, to allow airflow into and out of the nasal valve body 3191.1 to be controlled by the nasal valve 3190 during inhalation and exhalation through a user's nasal airway.

The sliding adjustment arrangement 3170 includes an adjustment flange 3171.1 formed on the oral valve body 3141.1, and two complemental clamping flanges 3174.1 formed on the nasal valve body 3191.1. A curved, slotted hole 3171.2 is formed in the adjustment flange 3171.1, and a clamping bolt 3174.2 passes through the two clamping flanges 3174.1 and the curved slotted hole 3171.2. Appropriately tensioning the nut of the clamping bolt 3174.2 can clamp the two clamping flanges 3174.1 to the adjustment flange to provide a friction force sufficient to fix the nasal valve body 3191.1 relative to the oral valve body 3141.1. Loosening the nut of the clamping bolt 3174.2 to relieve this friction force allows the nasal valve body 3191.1 to be moved relative to the oral valve body 3141.1 with the clamping bolt 3174.2 moving along the slotted hole 3171.2.

In this example, the nasal pillow connector 3144.1, with the integrally formed nasal pillow tubes 3144.2, is reversibly coupled directly to the nasal valve body 3191.1 with a clip or snap fit. It will be appreciated that other reversible coupling arrangements may also be utilised, as described above with respect to the valve arrangement described with reference to FIGS. 29A and 29B. In addition, in this example, the oral valve 3150 and the nasal valve 3190 both share the same configuration as the valve 1850 described above with reference to FIGS. 18A to 18D. It will be appreciated that, in other examples, one or both of the oral valve 3150 and the nasal valve 3190 may be provided with an alternative configuration.

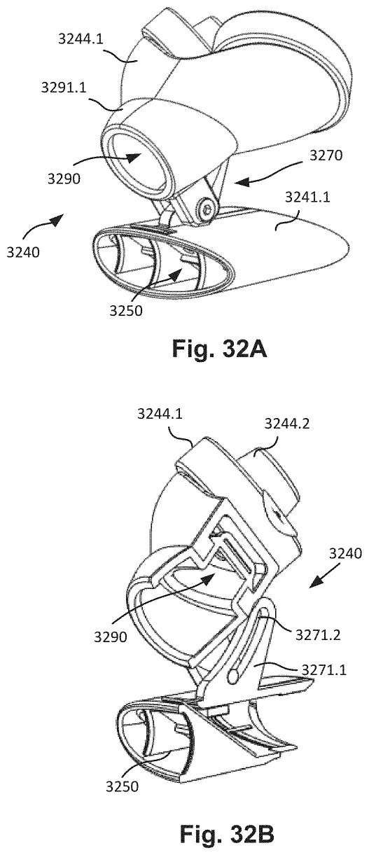

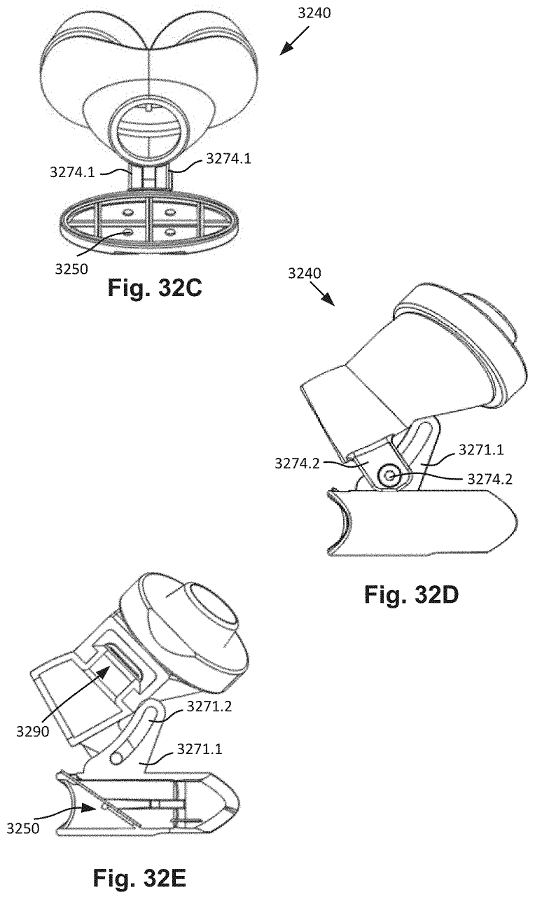

An example of such an alternative valve arrangement will now be described with reference to FIGS. 32A to 32D, which shows a modified version of the valve arrangement shown in FIGS. 31A and 31B. For ease of explanation, this explanation uses reference numerals similar to those used in the example of FIGS. 31A and 31B, albeit increased by 100, and such features will not therefore be described in further detail.

In this example, the oral valve 3250 shares the same general configuration as described above with respect to the valve arrangement 540 shown in FIGS. 5A to 5D, and the nasal valve 3290 shares the same general configuration as described above with respect to the valve arrangement shown in FIGS. 4A to 4D. In this example, similar to the valve members 843.1, 843.2 of the valve arrangement 840 depicted in FIGS. 8A to 8D, the valve members of both the oral valve 3250 and the nasal valve 3290 include one or more openings therein to allow airflow through the valve members when they are in a closed position. Thus, in this configuration, this allows exhalation to occur via the openings, which thereby introduces resistance during exhalation, in turn creating pressure in the oral and nasal cavities, maintaining a positive airway pressure within the patient's airway to maintain airway inflation which can help maintain the user's airway open. It will be appreciated that the discussion above with respect to various configurations of the valve members 843.1, 843.2 of the valve arrangement 840 depicted in FIGS. 8A to 8D applies equally to both the oral valve 3250 and the nasal valve 3290 in this example.

Also, in this example, the nasal pillow connector 3244.1, with the integrally formed nasal pillow tubes 3244.2, is reversibly coupled directly to the nasal valve body 3291.1 with a clip or snap fit. The nasal valve 3290 is located at the opening of the nasal valve body 3291.1 that snaps or clips into the lower opening of the nasal pillow connector 3244.1. It will be appreciated that other reversible coupling arrangements may also be utilised, as described above with respect to the valve arrangement described with reference to FIGS. 29A and 29B. It will also be appreciated that the nasal valve 3290 may be located at other positions in the nasal valve body 3291.1.

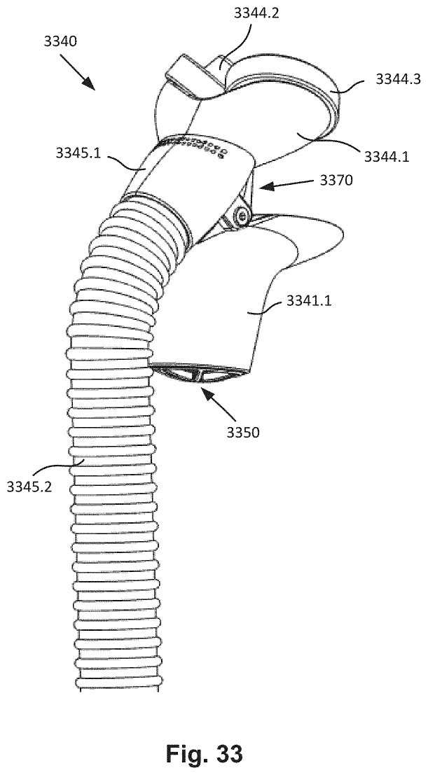

A further example of a valve arrangement will now be described with reference to FIG. 33, which shows a modified version of the valve arrangement shown in FIGS. 31A and 31B. For ease of explanation, this explanation uses reference numerals similar to those used in the example of FIGS. 31A and 31B, albeit increased by 400, and such features will not therefore be described in further detail.

In this example valve arrangement 3340, the nasal valve body 3191.1 of valve arrangement 3140 is replaced by a device connector 3345.1 coupled to a tube 3345.2 defining a device airway that supplies airflow from a positive airway pressure device. The device connector 3345 is mounted to the valve body 3341.1 via a sliding adjustment arrangement 3370, which has the same configuration as the sliding adjustment arrangements shown in FIGS. 31A to 32E, and is coupled to the nasal pillow connector 3344.1 using a suitable coupling arrangement, such as a clip, snap, friction or interference fit, or through a magnetic mechanism. The valve body 3341.1 is substantially the same as the oral valve body 3141.1 described above with reference to FIGS. 31A and 31B. It will be appreciated that the valve body 3341.1 may adopt an alternative configuration, such as the configuration of the oral valve body 3241.1 of the valve arrangement depicted in FIGS. 32A to 32E.

In this example, the device airway is in fluid communication with the nasal airway, allowing positive air pressure to be supplied to the user's nasal cavity. A number of device connector airway openings are provided in the device connector 3345.1 to control air pressure in the device connector airway. It will be appreciated that, in other examples, additional or alternative airway openings could be provided in the tube 3345.2. The airway openings could be used in conjunction with a nasal valve provided to assist in the device connector controlling nasal cavity air pressure during inhalation and exhalation.

Accordingly, it will be appreciated that in some embodiments, the oral appliance and connector can include first and second ports in communication with respective appliance intra-oral openings, which allow airflow, O₂ or other gases to be delivered intra-orally, and which also allow exhaled air to be sensed, for example for determining end tidal volume. Such communication could be direct, by having the port extend into the oral cavity, and/or could be via an appliance airway, for by providing a port in communication with an appliance airway extending to an intra-oral opening.

In one example, this is achieved using an ISO 5367-2014 compliant port, which can be attached to oxygen (or AIR) at a suitable flow rate, such as 6 litres/min, with or without a valve. This can provide sufficient airway pneumatic splinting for hospital or remote settings. A Luer lock port can also be provided that measures CO₂ end tidal volume. The connectors could be used in surgery, for example, for monitored sedation or Total Intravenous Anesthesia (TIVA) or narcotic infusions. The device can allow for deeper sedation without airway obstruction/hypoxia and reduce litigation for lack of awareness. Additionally, this can help prevent dental injury from biting on a Guedel airway or a laryngeal mask during removal. The arrangement could also be used for home or remote use, for example for COPD/OSA/Asthma sufferers.

In further examples, the ports can communicate with channels within the oral appliance, allowing O₂ to be delivered to the back of the mouth via a dedicated channel, separate to an air inhalation/exhalation channel. In this case additional devices can be added to the front of the appliance, for example to allow for additional equipment to be used, including, but not limited to, an Ambu bag for resuscitation, an in-line PEEP valve, a filter or a non-rebreathing oxygen mask.

In a further example, the oral appliance can be configured to provide access to the oral cavity, for example, to allow for insertion of instruments, as occurs during gastroscopy, endoscopy or the like. In this instance, an opening can be provided in the oral appliance, to retain the users mouth open, whilst also allowing delivery of O₂, or the like via respective channels.

This can help protect expensive instruments, and avoid injury to the patient. The configuration can move the oxygen deeply into the buccal/alveolar space on each side through a dedicated split airway and have wider dental anchoring to take the pressure off the (vulnerable) front teeth. In addition a Luer lock port can be provided to measure CO2 end tidal volume if required.

Accordingly, it will be appreciated that at least some of the above described examples provide valve arrangements that are configured to attach to the extra-oral opening of an oral appliance to provide a breathing assistance apparatus that can provide differential resistance to airflow during inhalation and exhalation. In particular, these arrangements can be used to provide differential resistance during inhalation and exhalation, with the levels of resistance being controlled through appropriate configurations of the valves, in turn allowing different levels of positive airway pressure to be generated within the user's airway during exhalation.

Adjustment of the characteristics of the valve arrangements, as well as different combinations of valve arrangements, such as oral only, nasal only, a combination of oral and nasal, or the like, can be used to control the resulting positive airway pressure, with this being performed based on user breathing characteristics to thereby optimise the airway pressure based on the requirements of each user.

Whilst the arrangement has been described with reference to the oral appliance of FIGS. 1A to 1E, it will be appreciated that this is not essential and similar arrangements could be implemented with any suitable oral appliance. For example, the airway arrangement of the oral appliance of FIGS. 1A to 1E, whilst preferred, is not intended to be limiting. Specifically, the valve arrangement described in more detail below can be used with any configuration of oral appliance that includes an airway to allow airflow via the oral cavity. For example, the airway could extend straight through the tubular body 131.1 and into a front of the oral cavity, thereby effectively providing an airway that passes directly between the lips and incisors. Alternatively, the airway could include allow airflow into the oral cavity along the entire oral cavity length, for example by using an open channel, or part of the oral cavity, for example using one or more openings provided along the channel length.

Additionally and/or alternatively, in certain embodiments, the valve arrangement can be used with an oral appliance with no airway, for example when the valve arrangement is used to control airflow via the user's nasal cavity. In this instance, the oral appliance can be in the form of a moulded bite member, with this effectively acting merely to support a valve arrangement outside the user's mouth. Particularly in the case of delivering positive air pressure to the user's nasal passage, this can represent a viable alternative to the use of a mask, which is the preferred arrangement for most traditional PAP techniques, but which user's tend to find uncomfortable.

Thus, it will be appreciated that the valve arrangement is intended to be used with a wide variety of oral appliance, and that reference to the appliance of FIGS. 1A to 1E, whilst beneficial, is not intended to be limiting.

It will also be appreciated that the above described arrangements can also be used in conjunction with other features. For example, the valve arrangement and/or the extra-oral opening can include a heat and/or moisture exchanger, such as a heat moisture exchange (HME) sponge, that controls the water and temperature content of the air being inhaled by exchanging heat and moisture with exhaled air. In one example, this can be provided within the extra-oral opening, with the valve being external to the extra-oral opening, or vice versa, to ensure there is sufficient available space to accommodate both the HME sponge and the valve, although this is not essential and alternatively by the valve and heat and/or moisture exchanger could be incorporated into the valve arrangement and/or the extra-oral opening.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

It will of course be realised that whilst the above has been given by way of an illustrative example of this invention, all such and other modifications and variations hereto, as would be apparent to persons skilled in the art, are deemed to fall within the broad scope and ambit of this invention as is herein set forth. It will be understood that the invention disclosed and defined in this specification extends to all alternative combinations of two or more of the individual features mentioned or evident from the text or drawings. All of these different combinations constitute various alternative aspects of the invention.

We claim:

1. A breathing assistance apparatus including:
a) an oral appliance having an appliance body shaped to be at least partially positioned within the oral cavity of a user; and,
b) a valve arrangement including:
i) a valve body including a valve airway in fluid communication with a user airway; and,
ii) a valve member positioned in the valve airway, the valve member being movable between first and second positions at least in part as a result of airflow through the valve airway and wherein the valve member at least partially obstructs the valve airway in the second position so that there is a differential resistance during inhalation and exhalation.

2.-3. (canceled)

4. Apparatus according to claim 1, including an appliance airway to allow airflow into and/or out of an oral cavity of the user; and a nasal airway to allow airflow into and/or out of a nasal cavity of the user, and wherein the valve arrangement includes:
a) an oral valve to control breathing via the oral cavity; and,
b) a nasal valve to control breathing via the nasal cavity.

5.-7. (canceled)

8. Apparatus according to 1, wherein the valve member includes one or more valve member openings to allow airflow through the valve member when the valve member is in the second position, and/or the valve body includes one or more openings to allow airflow through the valve body at least when the valve member is in the second position.

9.-12. (canceled)

13. Apparatus according to claim 1, wherein the valve member is biased at least one of:
a) into the first position by airflow through the valve airway;
b) into the second position by at least one of:
i) airflow through the valve airway; and,
ii) elasticity of the valve member, and,
e) into the first position through elastic deformation of the valve member during inhalation and elastically returns to the second position when the user is not inhaling.

14.-15. (canceled)

16. Apparatus according to claim 1, wherein the valve member is a silicone flap and/or engages the valve body to retain the valve member in the second position.

17. Apparatus according to claim 1, wherein the appliance body includes an extra-oral opening that extends between lips of the user and an appliance airway passing through the body to an intra-oral opening provided in the oral cavity to allow airflow into and/or out of a posterior region of the oral cavity, and the valve body optionally is coupled to the extra-oral opening through at least one of:
a) a friction fit;
b) an interference fit;
e) a clip fit; and,
d) magnetic engagement.

18. (canceled)

19. Apparatus according to claim 17, wherein the valve body includes at least one of:
a) a ring configured to be positioned external to and in abutment with the extra-oral opening, wherein the valve airway includes one or more valve airway openings through the ring and the valve member is optionally coupled to the ring so that at least part of the valve member is positioned between the ring and the extra-oral opening;
b) one or more guides to position the valve body relative to the extra-oral opening;
c) one or more tabs including lips adapted to engage a groove within the extra-oral openings to thereby couple the valve body to the extra-oral opening; and
d) a hollow tube having a substantially oval cross-section.

20. Apparatus according to claim 19, wherein valve member is coupled to the ring so that at least part of the valve member is positioned between the ring and the extra-oral opening.

21.-24. (canceled)

25. Apparatus according to 1, wherein the valve body includes a number of valve airway openings and wherein the valve member is adapted to obstruct selected ones of the valve airway openings, and/or the valve arrangement includes a plurality of valve members.

26. (canceled)

27. Apparatus according to claim 1, wherein the valve body includes at least one valve airway opening and a shoulder extending at least part way round the at least one valve airway opening, wherein the valve member at least partially obstructs the at least one valve airway opening, and wherein the valve member engages the shoulder in the second position to retain the valve member in the second position.

28. (canceled)

29. Apparatus according to claim 9, wherein the valve body includes one or more struts extending across the opening, and wherein the valve member engages the struts in the second position to retain the valve member in the second position.

30. Apparatus according to claim 1, wherein the valve body is coupled to a nasal pillow defining at least partially a nasal airway to allow airflow into and out of the nasal cavity of the user, wherein a nasal valve member is optionally positioned in the nasal airway, the nasal valve member being movable between first and second positions at least in part as a result of airflow through the nasal airway and wherein the nasal valve member at least partially obstructs the nasal valve airway in the second position so that there is a differential resistance during inhalation and exhalation via the nasal cavity.

31. Apparatus according to claim 11, wherein the nasal pillow is mounted on a nasal pillow connector body attachable to the valve body, and wherein the nasal pillow connector body optionally is at least one of:
a) movably mounted to the valve body to allow a relative position of the nasal pillow and valve body to be adjusted;
b) integrally formed with the nasal pillow from a rigid or semi-rigid material such as nylon, polyurethane or silicone rubber;
c) reversibly attachable through at least one of:
i) a friction fit;
ii) an interference fit;
iii) a clip fit; and,
iv) magnetic engagement, and,
d) attachable to the valve body through a nasal pillow connector stalk.

32.-35. (canceled)

36. Apparatus according to claim 11, wherein the valve body includes a nasal valve body and an oral valve body, the nasal pillow connector being directly attachable to the nasal valve body such that the nasal airway is at least partially defined by the nasal valve body, the nasal pillow connector and the nasal pillows, and wherein the nasal valve body and the oral valve body optionally are coupled to each other in an adjustable manner.

37.-39. (canceled)

40. Apparatus according to claim 1, wherein a device connector is coupled to the valve body, the device connector defining a device airway that supplies airflow from a positive airway pressure device, wherein the device airway is in fluid communication with the valve airway and/or a nasal airway, and wherein the device connector optionally includes a number of device connector airway openings to control air pressure in the device connector airway.

41.-43. (canceled)

44. Apparatus according to claim 1, wherein the appliance body defines at least two channels, each channel connecting an intra-oral opening to an extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space, and/or wherein the appliance body optionally includes a hollow lateral base extending inwardly from a hollow arcuate side wall, the extra-oral opening optionally being defined by a tubular body protruding forwardly from the arcuate side wall.

45. Apparatus according to claim 1, wherein the oral appliance includes at least one bite member coupled to the body, the bite member being positioned at least partially between the user's teeth and the body in use and/or mechanically engaging an inner surface of the hollow side wall to thereby couple the at least one bite member to the body.

46.-48. (canceled)

49. Apparatus according to claim 1, wherein the valve member is part of a valve that is removably mounted to the valve body to allow valve members to be interchanged, and/or wherein the valve member is mounted on a valve ring, and wherein the valve ring is biased into engagement with a valve seat within the valve body such that movement of the valve member controls resistance during inhalation and biasing of the valve ring against the valve seat controls resistance during exhalation.

50. Apparatus according to claim 1, wherein the valve arrangement includes a first valve mechanism to control resistance during inhalation and a second valve mechanism to provide resistance during exhalation, and/or wherein the valve arrangement includes:
a) a base that in use is attached to the valve body;
b) a spring; and,
c) a valve ring, the valve ring supporting a valve member movable so that the valve ring is open during inhalation and closed during exhalation, and wherein the valve ring is biased into engagement with a valve seat so that the valve is open during exhalation and closed during inhalation, and
wherein the base optionally is movably mounted to the valve body to allow a spring compression to be adjusted and control resistance during exhalation.

51. (canceled)

52. Apparatus according to claim 1, wherein the valve arrangement includes:
a) a first valve to control resistance to airflow via a user's oral cavity; and,
b) a second valve to control resistance to airflow via a user's nasal cavity.

53. Apparatus according to claim 1, wherein the apparatus includes a port in communication with at least one of an appliance airway and a valve airway, wherein in use the port is used to deliver at least one of gases and medication to a subject's airway, and wherein the port is optionally coupled to a delivery tube extending along an oral appliance airway.

54. (canceled)

55. Apparatus according to claim 53, wherein the apparatus includes a second port in communication with an intra-oral opening, wherein in use the port is used to sample exhaled air.

56. (canceled)

57. Apparatus according to claim 1, wherein the apparatus includes first and second ports in communication with respective appliance intra-oral openings and/or wherein the appliance body includes a tubular body configured to extend between lips of the user, and wherein the tubular body includes:
a) a first extra-oral opening in fluid communication with an appliance airway passing through the body to an intra-oral opening provided in the oral cavity to allow airflow into and/or out of a posterior region of the oral cavity; and,
b) a second extra-oral opening extending into the oral cavity of the user to provide access to the oral cavity, wherein the second extra-oral opening is configured to receive a surgical instrument.

58. (canceled)

59. A valve arrangement for use with an oral appliance having an appliance body shaped to be at least partially positioned within the oral cavity of a user, the valve arrangement including:
a) a valve body including a valve airway in fluid communication with a user airway; and,
b) a valve member positioned in the valve airway, the valve member being movable between first and second positions at least in part as a result of airflow through the valve airway and wherein the valve member at least partially obstructs the valve airway in the second position so that there is a differential resistance during inhalation and exhalation.

60.-61. (canceled)

62. A method of configuring a breathing assistance apparatus for a user, the breathing assistance apparatus including:
a) an oral appliance having an appliance body shaped to be at least partially positioned within the oral cavity of a user; and,
b) a valve arrangement including:
i) a valve body including a valve airway in fluid communication with a user airway; and,
ii) a valve member positioned in the valve airway, the valve member being movable between first and second positions at least in part as a result of airflow through the valve airway and wherein the valve member at least partially obstructs the valve airway in the second position so that there is a differential resistance during inhalation and exhalation, the method including
(1) determining breathing characteristics of a user; and,
(2) configuring the breathing assistance apparatus at least partially in accordance with the breathing characteristics of the user.

63. (canceled)

64. A method according to claim 62, wherein the method includes configuring the breathing assistance apparatus by configuring the valve arrangement to generate a positive airway pressure in a user airway by at least one of:
a) restricting airflow during exhalation via the oral cavity; and,
b) restricting airflow during exhalation via the nasal cavity.

65. A method according to claim 62, wherein the method includes configuring the breathing assistance apparatus to generate a positive airway pressure in a user airway by connecting a device airway to a device connector coupled to the valve body, the device connector defining a device airway that supplies airflow from a positive airway pressure device, and wherein the device airway is in fluid communication with at least one of:
a) the valve airway; and,
b) a nasal airway.

66. A method according to claim 62, wherein the method includes progressively introducing positive airway pressure in a user airway by at least one of:
a) restricting airflow during exhalation via the oral cavity;
b) restricting airflow during exhalation via the nasal cavity;
c) restricting airflow during exhalation via the oral and nasal cavities;
d) supplying positive airway pressure from a positive airway pressure device; and,
e) supplying positive airway pressure from a positive airway pressure device and restricting airflow during exhalation via the oral cavity.
